SEQUENCE LISTING (48 sequences)

SEQ ID NO: 1 - protein, 17 aa, Artificial Sequence
  Neo-epitope peptide sequence NBH-222 from calprotectin
KLGHPDTLNQGEFKELV

SEQ ID NO: 2 - protein, 17 aa, Artificial Sequence
  Neo-epitope peptide sequence NBH-223 from calprotectin
RKDLQNFLKKENKNEKV

SEQ ID NO: 3 - protein, 18 aa, Artificial Sequence
  Neo-epitope peptide sequence from calprotectin
RKDLQNFLKKENKNEKVI

SEQ ID NO: 4 - protein, 15 aa, Artificial Sequence
  Neo-epitope peptide sequence from calprotectin
EHIMEDLDTNADKQL

SEQ ID NO: 5 - protein, 25 aa, Artificial Sequence
  Neo-epitope peptide sequence NBH-224 from calprotectin
SHEKMHEGDEGPGHHHKPGLGEGTP

SEQ ID NO: 6 - protein, 11 aa, Artificial Sequence
  Neo-epitope peptide sequence NBH-225 from calprotectin
YRDDLKKLLET

SEQ ID NO: 7 - protein, 13 aa, Artificial Sequence
  Neo-epitope peptide sequence NBH-226 calprotectin
WFKELDINTDGAV

SEQ ID NO: 8 - protein, 11 aa, Artificial Sequence
  N-terminus elongated NBH-222 peptide
VKLGHPDTLNQ

SEQ ID NO: 9 - protein, 9 aa, Artificial Sequence
  N-terminus truncated NBH-222 peptide
LGHPDTLNQ

SEQ ID NO: 10 - protein, 11 aa, Artificial Sequence
  C-terminus elongated NBH-222 peptide
LNQGEFKELVR

SEQ ID NO: 11 - protein, 9 aa, Artificial Sequence
  C-terminus truncated NBH-222 peptide
LNQGEFKEL

SEQ ID NO: 12 - protein, 10 aa, Artificial Sequence
  Synthetic peptide sequence to raise the monoclonal antibody
KLGHPDTLNQ

SEQ ID NO: 13 - protein, 17 aa, Artificial Sequence
  Light chain complementary determining region CDR-L1 sequence
KSSQSLLNSGNQKNYLA

SEQ ID NO: 14 - protein, 7 aa, Artificial Sequence
  Light chain complementary determining region CDR-L2 sequence
GASTRES

SEQ ID NO: 15 - protein, 9 aa, Artificial Sequence
  Light chain complementary determining region CDR-L3 sequence
LNDHSYPYT

SEQ ID NO: 16 - protein, 5 aa, Artificial Sequence
  Heavy chain complementary determining region CDR-H1 sequence
DHVIN

SEQ ID NO: 17 - protein, 17 aa, Artificial Sequence
  Heavy chain complementary determining region CDR-H2 sequence
EIYPGSGSTYYNEKFKG

SEQ ID NO: 18 - protein, 3 aa, Artificial Sequence
  Heavy chain complementary determining region CDR-H3 sequence
FAY

SEQ ID NO: 19 - protein, 80 aa, Artificial Sequence
  Light chain complementary determining regions and framework sequence
KSSQSLLNSGNQKNYLAWYQQKPGQPPKLLIYGASTRESGVPDRFTGSGSGTDFTLTISSVQAEDLAVYYCLNDHSYPYT

SEQ ID NO: 20 - protein, 71 aa, Artificial Sequence
  Heavy chain complementary determining regions and framework sequence
DHVINWVRQRTGQGLEWIGEIYPGSGSTYYNEKFKGKATLTADKSSNTAYMQLSSLTSEDSAVYFCAWFAY

SEQ ID NO: 21 - protein, 113 aa, Artificial Sequence
  Light chain variable region sequence
DIVMTQSPSSLSVSAGEKVTMSCKSSQSLLNSGNQKNYLAWYQQKPGQPPKLLIYGASTRESGVPDRFTGSGSGTDFTLTISSVQAEDLAVYYCLNDHSYPYTFGGGTKLEIK

SEQ ID NO: 22 - protein, 112 aa, Artificial Sequence
  Heavy chain variable region sequence
QVQLQQSGPELVKPGASVKMSCKASGYTFTDHVINWVRQRTGQGLEWIGEIYPGSGSTYYNEKFKGKATLTADKSSNTAYMQLSSLTSEDSAVYFCAWFAYWGQGTLVTVSA

SEQ ID NO: 23 - protein, 10 aa, Artificial Sequence
  Biotinylated peptide of SEQ ID NO 12
  MOD_RES 10: Gln is biotinylated, optionally via a linker
KLGHPDTLNQ

SEQ ID NO: 24 - protein, 13 aa, Artificial Sequence
  Immunogenic peptide modified with Keyhole Limpet Hemocyanin
  MOD_RES 13: Cys is bound to Keyhole Limpet Hemocyanin
KLGHPDTLNQGGC

SEQ ID NO: 25 - protein, 454 aa, Artificial Sequence
  Monoclonal antibody heavy chain sequence, mouse IgG1 isotype
MEWRIFLFILSGTAGVHSQVQLQQSGPELVKPGASVKMSCKASGYTFTDHVINWVRQRTGQGLEWIGEIYPGSGSTYYNEKFKGKATLTADKSSNTAYMQLSSLTSEDSAVYFCAWFAYWGQGTLVTVSAAKTTPPSVYPLAPGSAAQTNSMVTLGCLVKGYFPEPVTVTWNSGSLSSGVHTFPAVLQSDLYTLSSSVTVPSSTWPSETVTCNVAHPASSTKVDKKIVPRDCGCKPCICTVPEVSSVFIFPPKPKDVLTITLTPKVTCVVVDISKDDPEVQFSWFVDDVEVHTAQTQPREEQFNSTFRSVSELPIMHQDWLNGKEFKCRVNSAAFPAPIEKTISKTKGRPKAPQVYTIPPPKEQMAKDKVSLTCMITDFFPEDITVEWQWNGQPAENYKNTQPIMDTDGSYFVYSKLNVQKSNWEAGNTFTCSVLHEGLHNHHTEKSLSHSPGK

SEQ ID NO: 26 - protein, 240 aa, Artificial Sequence
  Monoclonal antibody light chain sequence, mouse kappa isotype
MESQTQVLISLLFWVSGACGDIVMTQSPSSLSVSAGEKVTMSCKSSQSLLNSGNQKNYLAWYQQKPGQPPKLLIYGASTRESGVPDRFTGSGSGTDFTLTISSVQAEDLAVYYCLNDHSYPYTFGGGTKLEIKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 27 - protein, 11 aa, Artificial Sequence
  Biotinylated antigen sequence
  MOD_RES 11: Lys is biotinylated
KLGHPDTLNQK

SEQ ID NO: 28 - protein, 12 aa, Artificial Sequence
  Neo-epitope peptide sequence from calprotectin
MEDLDTNADKQL

SEQ ID NO: 29 - protein, 16 aa, Artificial Sequence
  Neo-epitope peptide sequence from calprotectin
EHIMEDLDTNADKQLS

SEQ ID NO: 30 - protein, 10 aa, Artificial Sequence
  Neo-epitope peptide sequence from calprotectin
SQLERNIETI

SEQ ID NO: 31 - protein, 16 aa, Artificial Sequence
  Neo-epitope peptide sequence from calprotectin
KLGHPDTLNQGEFKEL

SEQ ID NO: 32 - protein, 24 aa, Artificial Sequence
  Neo-epitope peptide sequence from calprotectin
HEKMHEGDEGPGHHHKPGLGEGTP

SEQ ID NO: 33 - protein, 27 aa, Artificial Sequence
  Neo-epitope peptide sequence from calprotectin
WASHEKMHEGDEGPGHHHKPGLGEGTP

SEQ ID NO: 34 - protein, 20 aa, Artificial Sequence
  Neo-epitope peptide sequence from calprotectin
HEGDEGPGHHHKPGLGEGTP

SEQ ID NO: 35 - protein, 13 aa, Artificial Sequence
  Neo-epitope peptide sequence from calprotectin
MEDLDTNADKQLS

SEQ ID NO: 36 - protein, 19 aa, Artificial Sequence
  Neo-epitope peptide sequence from calprotectin
SHEKMHEGDEGPGHHHKPG

SEQ ID NO: 37 - protein, 10 aa, Artificial Sequence
  Synthetic peptide
IDVYHKYSLI

SEQ ID NO: 38 - protein, 114 aa, Homo sapiens
  S100A9 protein Sequence
MTCKMSQLERNIETIINTFHQYSVKLGHPDTLNQGEFKELVRKDLQNFLKKENKNEKVIEHIMEDLDTNADKQLSFEEFIMLMARLTWASHEKMHEGDEGPGHHHKPGLGEGTP

SEQ ID NO: 39 - protein, 5 aa, Artificial Sequence
  1..5: S100A9 protein fragment
MTCKM

SEQ ID NO: 40 - protein, 9 aa, Artificial Sequence
  16..24: S100A9 protein fragment
INTFHQYSV

SEQ ID NO: 41 - protein, 12 aa, Artificial sequence
  S100A9 protein fragment
FEEFIMLMARLT

SEQ ID NO: 42 - protein, 18 aa, Artificial sequence
  S100A9 protein fragment
SHEKMHEGDEGPGHHHKP

SEQ ID NO: 43 - protein, 7 aa, Artificial sequence
  S100A9 protein fragment
GLGEGTP

SEQ ID NO: 44 - protein, 93 aa, Homo sapiens
  S100A8 protein sequence
MLTELEKALNSIIDVYHKYSLIKGNFHAVYRDDLKKLLETECPQYIRKKGADVWFKELDINTDGAVNFQEFLILVIKMGVAAHKKSHEESHKE

SEQ ID NO: 45 - protein, 12 aa, Artificial Sequence
  S100A8 protein fragment
MLTELEKALNSI

SEQ ID NO: 46 - protein, 7 aa, Artificial Sequence
  S100A8 protein fragment
KGNFHAV

SEQ ID NO: 47 - protein, 13 aa, Artificial Sequence
  S100A8 protein fragment
ECPQYIRKKGADV

SEQ ID NO: 48 - protein, 27 aa, Artificial Sequence
  S100A8 protein fragment
NFQEFLILVIKMGVAAHKKSHEESHKE

(19) United States
(12) Patent Application Publication
(10) Pub. No.: US 2023/0184787 A1
(43) Pub. Date: Jun. 15, 2023

(54) Calprotectin Assay

(71) Applicant: Nordic Bioscience A/S, Herlev (DK)

(72) Inventors: Joachim Høg Mortensen, Hundige Strand (CN); Dovile Sinkeviciute, København N (DK); Tina Manon-Jensen, Brøndby (DK); Morten Asser Karsdal, København Ø (DK); Anne-Christine Bay-Jensen, København S (DK); Signe Holm Nielsen, Frederiksberg (DK); Annika Hummersgaard Hansen, Vanløse (DK); Jannie Marie Bülow Sand, Malov (DK); Diana Julie Leeming, Klampenborg (DK); Nicholas Willumsen, Dyssegard (DK); Christina Jensen, København N (DK)

(73) Assignee: Nordic Bioscience A/S, Herlev (DK)

(21) Appl. No.: 17/925,005

(22) PCT Filed: May 12, 2021
(86) PCT No.: PCT/EP2021/062753

§ 371 Date: Nov. 13, 2022

(30) Foreign Application Priority Data
May 13, 2020 (GB) 2007087.6
Jan. 21, 2021 (GB) 2100902.2

(51) Int. Cl. G01N 33/68; C07K 16/18

(52) U.S. Cl. CPC G01N 33/6893; C07K 16/18; G01N 2333/4727; G01N 2800/56; G01N 2800/7095; G01N 2470/10

(57) ABSTRACT

Disclosed herein are immunoassay methods for detecting and quantifying human neutrophil elastase (HNE) generated fragments of calprotectin in a biofluid sample; and monoclonal antibodies and assay kits for use in such methods. The methods may be used for detecting, monitoring and/or determining the status or severity of a disease characterized by or exhibiting inflammation in a patient.

### FIELD OF THE INVENTION

The present invention relates to immunoassay methods for detecting and, preferably, quantifying human neutrophil elastase (HNE) generated fragments of calprotectin in a biofluid sample, and to the use of such methods for detecting, monitoring and/or determining the status or severity of a disease characterized by or exhibiting inflammation, such as but not limited to an inflammatory driven disease, in a patient. The present invention also relates to monoclonal antibodies and assay kits for use in such methods.

### BACKGROUND

Calprotectin is expressed intracellularly, especially in neutrophil granulocytes, and comprises around 60% of neutrophil granulocytes total cytosolic matter (1). Calprotectin is a ligand for TLR receptors and is relatively protease resistant (2). Neutrophil granulocytes are specialized hematopoietic cells from the myeloid cell lineage and are a sub-group of cells from the innate immune system. Neutrophil granulocytes serve as a first in line of defense against pathogen and bacteria invasion, and are thus also highly expressed in inflamed tissue (3-5).

Calprotectin can be measured in the faeces (faecal-calprotectin) of patients with inflammatory bowel disease (IBD) (1), where faecal-calprotectin has proven to be a robust sandwich-ELISA biomarker for distinguishing IBD patients from patients with irritable bowel syndrome (6-8). As serum/plasma samples are much easier to handle than faecal samples, and as faecal consistency has also proven to affect faecal-calprotectin levels with high day to day and even stool to stool variation, many of the faecal-calprotectin assays have also been tested to see whether they could also be applied to serum/plasma samples. However, calprotectin has a very short half-life in plasma (5 hours) (9), and the current available data in regards to serum/plasma calprotectin and its utility as an applicable biomarker for IBD is controversial (10-15).

Activation of neutrophil granulocytes results in the production of neutrophil extracellular traps (NETs), where the cells secrete all their nuclei and cytosolic matter in an attempt to trap and impair e.g. invading bacteria (16). Calprotectin and human neutrophil elastase (HNE) will be secreted into the inflamed tissue, as a result of NET formation (17). Consequently, the tissue and extracellular matrix (ECM) and other secreted proteins will be degraded by HNE, which will result in the generation of neo-epitope containing protein fragments (18-19). In addition, HNE has also proven be related to tissue inflammation in IBD²⁰. Neo-epitope containing protein fragments of collagen degradation and formation have been demonstrated to be measured in the serum from IBD patients and pre-clinical models and are associated with clinical disease parameters (18, 21-25).

Inflammation induced release of calprotectin and HNE also occurs in other diseases. Chronic obstructive pulmonary disease (COPD) and idiopathic pulmonary fibrosis (IPF) are characterized by extensive inflammation and remodeling of the extracellular matrix (ECM) and this potentially leads to a severe decline in lung function over time (34,35). Neutrophils are highly abundant in a COPD-affected lung and cause persistent tissue damage (36). IPF patients experience similar persistent injury through fibrotic changes, where an increase of HNE release has been seen in bronchoalveolar lavage fluid for IPF patients (37). Neutrophils are cellular responders to inflammation and able to release both the protease NE and the protein calprotectin (38).

Inflammation also predisposes to the development of cancer and promotes all stages of tumorigenesis. Cancer cells, as well as surrounding stromal and inflammatory cells, engage in well-orchestrated reciprocal interactions to form an inflammatory tumor microenvironment (TME) (39). Immune checkpoint inhibitors, such as but not limited to anti-PD-1 therapies, are a class of drug used to treat cancers such as metastatic melanoma. However, peripheral biomarkers associated with response and resistance to anti-PD-1 therapies and other such immune checkpoint inhibitors in metastatic melanoma patients represent an unmet medical need. High neutrophil activity is also associated with immune checkpoint inhibitor failure, partially due to release of NETs which have been shown to protect tumor cells against cytotoxic attacks. As described above, HNE and calprotectin are the major NETs constituents.

### SUMMARY OF THE INVENTION

The present inventors theorized that neo-epitope containing fragments of calprotectin generated by HNE could be a measure of active local tissue inflammation and active leucocytes including neutrophils, rather than systemic inflammation or circulating leucocytes. Furthermore, the present inventors have now developed a robust and reliable immunoassay for detecting and quantifying HNE-generated neo-epitope containing fragments of calprotectin in biofluids such as serum and plasma, and have demonstrated the use of said immunoassay in evaluating inflammation and disease activity in diseases such as IBD, COPD, IPF, metastatic melanoma, SCLC NSCLC, rheumatoid arthritis, ankylosing spondylitis, psoriasis, psoriasis arthritis and osteoarthritis.

Accordingly, in a first aspect the present invention relates to an immunoassay method for detecting an HNE-generated fragment of calprotectin, said method comprising contacting a human biofluid sample with a monoclonal antibody that specifically recognises and binds to an HNE-generated neo-epitope consisting of an N-terminus or C-terminus sequence of the HNE-generated fragment of calprotectin, and detecting binding between the monoclonal antibody and peptides in the sample.

Preferably, the detection is quantitative, and the method further comprises determining the amount of binding between said monoclonal antibody and peptides in the sample.

In preferred embodiments, the method is an immunoassay method for detecting and/or monitoring the progress of and/or determining the status or severity of a disease in a patient, wherein the disease is a disease characterized by or exhibiting inflammation, the method comprising contacting a biofluid sample obtained from said patient with the monoclonal antibody, detecting and determining the amount of binding between the monoclonal antibody and peptides in the sample, and correlating said amount of binding with values associated with normal healthy subjects and/or with values associated with a known status or severity of the disease and/or with values obtained from said patient at a previous time point and/or with a predetermined cut-off value.

The disease may be an inflammatory driven disease, such as for example inflammatory bowel disease (IBD), rheumatoid arthritis, psoriasis, psoriasis arthritis, ankylosing spondylitis, osteoarthritis, Sjögrens syndrome, or lupus.

In a preferred embodiment, the disease is inflammatory bowel disease (IBD). In particular, the immunoassay method may be a method for detecting and/or monitoring the progress of and/or determining the status or severity of types of inflammatory bowel disease (IBD) such as ulcerative colitis (UC) and/or Crohn's disease (CD).

In another preferred embodiment, the disease is rheumatoid arthritis, ankylosing spondylitis, psoriasis, psoriasis arthritis or osteoarthritis.

In other preferred embodiments, the disease may be chronic obstructive pulmonary disease (COPD), idiopathic pulmonary fibrosis (IPF) or asthma.

In yet other embodiments the disease may be a cancer. The cancer may for example be a breast, prostate, lung, gastric, colorectal, pancreatic, melanoma, ovarian, kidney, head & neck, bladder cancer. The cancer may for example be a metastatic cancer. In particular embodiments, the cancer may for example be metastatic melanoma, small cell lung cancer (SCLC) or non-small cell lung cancer (NSCLC).

Where the method is a method for determining the status or severity of a disease, the method may for example be for assessing whether the disease is active or in remission; or for assessing a likely period of patient survival or progression-free survival; or for assessing a likely response to a medical intervention, such as a likely period of patient survival or progression-free survival with treatment with one or more drugs (such as one or more chemotherapeutic agents (i.e. cytotoxic agents) and/or immune checkpoint inhibitors).

The monoclonal antibody may specifically recognise and bind to an HNE-generated neo-epitope consisting of an N-terminus or C-terminus sequence of any HNE-generated fragment of calprotectin, such as any of the peptides listed in Table 2, infra. However, in certain preferred embodiments, the monoclonal antibody specifically recognises and binds to an N-terminus or C-terminus sequence of a peptide selected from one of the following HNE-generated fragments of calprotectin:

(SEQ ID NO: 1)
KLGHPDTLNQGEFKELV
(also referred to herein as “NBH-222”)
(SEQ ID NO: 2)
RKDLQNFLKKENKNEKV (“NBH-223”)
(SEQ ID NO: 3)
RKDLQNFLKKENKNEKVI
(SEQ ID NO: 4)
EHIMEDLDTNADKQL
(SEQ ID NO: 5)
SHEKMHEGDEGPGHHHKPGLGEGTP (“NBH-224”)
(SEQ ID NO: 6)
YRDDLKKLLET (“NBH-225”)
(SEQ ID NO: 7)
WFKELDINTDGAV (“NBH-226”)

In a particular preferred embodiment, the monoclonal antibody specifically recognises and binds to an N-terminus or C-terminus sequence of the peptide

(SEQ ID NO: 1)
KLGHPDTLNQGEFKELV (“NBH-222”)

In certain preferred embodiments, the monoclonal antibody specifically recognises and binds to said N-terminus sequence of the HNE-generated fragment of calprotectin. Preferably, said monoclonal antibody does not specifically recognise or bind to an N-extended elongated version of said N-terminus amino acid sequence or an N-truncated shortened version of said N-terminus amino acid sequence. In this regard “N-extended elongated version of said N-terminus amino acid sequence” means one or more amino acids extending beyond the N-terminus of the sequence. Similarly, “N-truncated shortened version of said N-terminus amino acid sequence” means one or more amino acids removed from the N-terminus of the sequence. Thus, for example, where the monoclonal antibody specifically recognises and binds to an N-terminus sequence of the peptide KLGHPDTLNQGEFKELV (“NBH-222”) (SEQ ID NO: 1) an “N-extended elongated version” would be VKLGHPDTLNQ . . . (SEQ ID NO: 8), and an “N-truncated shortened version” would be LGHPDTLNQ . . . (SEQ ID NO: 9).

In certain other embodiments, the monoclonal antibody specifically recognises and binds to said C-terminus sequence of the HNE-generated fragment of calprotectin, wherein preferably said monoclonal antibody does not specifically recognise or bind to a C-extended elongated version of said C-terminus amino acid sequence or a C-truncated shortened version of said C-terminus amino acid sequence. In this regard “C-extended elongated version of said C-terminus amino acid sequence” means one or more amino acids extending beyond the C-terminus of the sequence. Similarly, “C-truncated shortened version of said C-terminus amino acid sequence” means one or more amino acids removed from the C-terminus of the sequence. Thus, for example, where the monoclonal antibody specifically recognises and binds to a C-terminus sequence of the peptide KLGHPDTLNQGEFKELV (“NBH-222”) (SEQ ID NO: 1) an “C-extended elongated version” would be . . . LNQGEFKELVR (SEQ ID NO: 10), and an “C-truncated shortened version” would be . . . LNQGEFKEL (SEQ ID NO: 11).

The monoclonal antibody is preferably a monoclonal antibody raised against a synthetic peptide comprising said N-terminus or C-terminus sequence. Thus, for example, where the monoclonal antibody specifically recognises and binds to an N-terminus sequence of the peptide KLGHPDTLNQGEFKELV (“NBH-222”) (SEQ ID NO: 1), the monoclonal antibody may be a monoclonal antibody raised against a synthetic peptide having the sequence KLGHPDTLNQ (SEQ ID NO: 12) such that the monoclonal antibody specifically recognises and binds to an N-terminus sequence of the peptide KLGHPDTLNQGEFKELV (SEQ ID NO: 1). Suitable exemplary protocols for raising a monoclonal antibody against a synthetic peptide are described in the Examples infra.

In certain exemplary embodiments, where the monoclonal antibody specifically recognises and binds to an N-terminus sequence of the peptide KLGHPDTLNQGEFKELV (“NBH-222”) (SEQ ID NO: 1), and preferably does not specifically recognise or bind to an N-extended elongated version of said N-terminus amino acid sequence or an N-truncated shortened version of said N-terminus amino acid sequence, the monoclonal antibody may preferably comprise one or more complementarity-determining regions (CDRs) selected from:

CDR-L1:
(SEQ ID NO: 13)
KSSQSLLNSGNQKNYLA
CDR-L2:
(SEQ ID NO: 14)
GASTRES
CDR-L3:
(SEQ ID NO: 15)
LNDHSYPYT
CDR-H1:
(SEQ ID NO: 16)
DHVIN
CDR-H2:
(SEQ ID NO: 17)
EIYPGSGSTYYNEKFKG
CDR-H3:
(SEQ ID NO: 18)
FAY

Preferably the monoclonal antibody comprises at least 2, 3, 4, 5 or 6 of the above listed CDR sequences.

Preferably the monoclonal antibody has a light chain variable region comprising the CDR sequences:

CDR-L1:
(SEQ ID NO: 13)
KSSQSLLNSGNQKNYLA
CDR-L2:
(SEQ ID NO: 14)
GASTRES
and
CDR-L3:
(SEQ ID NO: 15)
LNDHSYPYT.

Preferably the monoclonal antibody has a light chain that comprises framework sequences between the CDRs, wherein said framework sequences are substantially identical or substantially similar to the framework sequences between the CDRs in the light chain sequence below (in which the CDRs are shown in bold and underlined, and the framework sequences are shown in italics)

(SEQ ID NO: 19)
KSSQSLLNSGNQKNYLAWYQQKPGQPPKLLIYGASTRESGVPDRFTGSG
SGTDFTLTISSVQAEDLAVYYCLNDHSYPYT

Preferably the monoclonal antibody has a heavy chain variable region comprising the CDR sequences:

CDR-H1:
(SEQ ID NO: 16)
DHVIN
CDR-H2:
(SEQ ID NO: 17)
EIYPGSGSTYYNEKFKG
and
CDR-H3:
(SEQ ID NO: 18)
FAY.

Preferably the monoclonal antibody has a heavy chain that comprises framework sequences between the CDRs, wherein said framework sequences are substantially identical or substantially similar to the framework sequences between the CDRs in the heavy chain sequence below (in which the CDRs are shown in bold and underlined, and the framework sequences are shown in italics)

(SEQ ID NO: 20)
DHVINWVRQRTGQGLEWIGEIYPGSGSTYYNEKFKGKATLTADKSSNTA
YMQLSSLTSEDSAVYFCAWFAY

Preferably, the monoclonal antibody comprises the light chain variable region sequence:

(SEQ ID NO: 21)
DIVMTQSPSSLSVSAGEKVTMSCKSSQSLLNSGNQKNYLAWYQQKPGQP
PKLLIYGASTRESGVPDRFTGSGSGTDFTLTISSVQAEDLAVYYCLNDH
SYPYTFGGGTKLEIK
(CDRs bold and underlined; Framework sequences in italics)

and/or the heavy chain variable region sequence:

(SEQ ID NO: 22)
QVQLQQSGPELVKPGASVKMSCKASGYTFTDHVINWVRQRTGQGLEWIG
EIYPGSGSTYYNEKFKGKATLTADKSSNTAYMQLSSLTSEDSAVYFCAW
FAYWGQGTLVTVSA
(CDRs bold and underlined; Framework sequences in italics)

The biofluid sample may be any type of biofluid, such as for example blood, urine, synovial fluid, serum, bronchoalveolar lavage fluid (BALF), or plasma. In preferred embodiments, however, the biofluid sample is plasma or serum.

The immunoassay may be, but is not limited to, a competitive assay or a sandwich assay. Similarly, the immunoassay may be, but is not limited to, an enzyme immunoassay (EIA) or a radioimmunoassay. Preferably, the immunoassay is competitive assay. Preferably, the immunoassay is an enzyme-linked immunosorbent assay (ELISA), such as in particular a competitive ELISA.

In a second aspect, the present invention relates to a monoclonal antibody that specifically recognises and binds to an HNE-generated neo-epitope consisting of an N-terminus or C-terminus sequence of an HNE-generated fragment of calprotectin. The monoclonal antibody is suitable for use in an immunoassay according to the first aspect of the invention, and preferred and other optional embodiments of the monoclonal antibody according to the second aspect of the invention will be apparent from the discussion supra of the monoclonal antibodies for use the in the first aspect of the invention and preferred and other optional embodiments thereof.

In a third aspect, the present invention relates to an immunoassay kit comprising a monoclonal antibody according to the second aspect of the invention, and at least one of:
a streptavidin coated well plate;
a biotinylated peptide comprising said N-terminus or C-terminus sequence linked to biotin;
a secondary antibody for use in a sandwich immunoassay
a calibrator peptide comprising said N-terminus or C-terminus sequence;
an antibody biotinylation kit;
an antibody HRP labelling kit; and
an antibody radiolabelling kit.

In a preferred embodiment, the immunoassay kit comprises the monoclonal antibody according to the second aspect of the invention, and one, two or all of:
a streptavidin coated well plate;
a biotinylated peptide comprising said N-terminus or C-terminus sequence linked to biotin; and
a calibrator peptide comprising said N-terminus or C-terminus sequence.

Where, for example, where the monoclonal antibody binds to an N-terminus sequence of the peptide KLGHPDTLNQGEFKELV (“NBH-222”) (SEQ ID NO: 1) a suitable biotinylated peptide may be a peptide having the sequence KLGHPDTLNQ-L-Biotin (SEQ ID NO: 23), wherein L is an optional linker. Similarly, where the monoclonal antibody binds to an N-terminus sequence of the peptide KLGHPDTLNQGEFKELV (“NBH-222”) (SEQ ID NO: 1) a suitable calibrator peptide may be a peptide having the sequence KLGHPDTLNQ (SEQ ID NO: 12).

### Definitions

As used herein, the terms “peptide” and “polypeptide” are used synonymously.

As used herein the term “monoclonal antibody” refers to both whole antibodies and to fragments thereof that retain the binding specificity of the whole antibody, such as for example a Fab fragment, Fv fragment, or other such fragments known to those skilled in the art. Antibodies which retain the same binding specificity may contain the same complementarity-determining regions (CDR). The CDR of an antibody can be determined using methods known in the art such as that described by Kabat et al. (33).

Antibodies can be generated from B cell clones as described in the examples. The isotype of the antibody can be determined by ELISA specific for human IgM, IgG or IgA isotype, or human IgG1, IgG2, IgG3 or IgG4 subclasses. Other suitable methods can be used to identify the isotype.

The amino acid sequence of the antibodies generated can be determined using standard techniques. For example, RNA can be isolated from the cells, and used to generate cDNA by reverse transcription. The cDNA is then subjected to PCR using primers which amplify the heavy and light chains of the antibody. For example, primers specific for the leader sequence for all VH (variable heavy chain) sequences can be used together with primers that bind to a sequence located in the constant region of the isotype which has been previously determined. The light chain can be amplified using primers which bind to the 3′ end of the Kappa or Lambda chain together with primers which anneal to the V kappa or V lambda leader sequence. The full length heavy and light chains can be generated and sequenced.

As used herein, the framework amino acid sequences between the CDRs of an antibody are “substantially identical” or “substantially similar” to the framework amino acid sequences between the CDRs of another antibody if they have at least 70%, 80%, 90% or at least 95% similarity or identity. The similar or identical amino acids may be contiguous or non-contiguous. The framework sequences may contain one or more amino acid substitutions, insertions and/or deletions. Amino acid substitutions may be conservative, by which it is meant the substituted amino acid has similar chemical properties to the original amino acid. A skilled person would understand which amino acids share similar chemical properties. For example, the following groups of amino acids share similar chemical properties such as size, charge and polarity: Group 1 Ala, Ser, Thr, Pro, Gly; Group 2 Asp, Asn, Glu, Gln; Group 3 His, Arg, Lys; Group 4 Met, Leu, Ile, Val, Cys; Group 5 Phe Thy Trp.

A program such as the CLUSTAL program to can be used to compare amino acid sequences. This program compares amino acid sequences and finds the optimal alignment by inserting spaces in either sequence as appropriate. It is possible to calculate amino acid identity or similarity (identity plus conservation of amino acid type) for an optimal alignment. A program like BLASTx will align the longest stretch of similar sequences and assign a value to the fit. It is thus possible to obtain a comparison where several regions of similarity are found, each having a different score. Both types of analysis are contemplated in the present invention. Identity or similarity is preferably calculated over the entire length of the framework sequences.

As used herein, the term “N-terminus” refers to the extremity of a polypeptide, i.e. at the N-terminal end of the polypeptide, and is not to be construed as meaning in the general direction thereof. Likewise, the term “C-terminus” refers to the extremity of a polypeptide, i.e. at the C-terminal end of the polypeptide, and is not to be construed as meaning in the general direction thereof.

As used herein the term, the term “competitive immunoassay” refers to an immunoassay in which the target peptide present in a sample (if any) competes with known amount of target of peptide (which, for example, is bound to a fixed substrate or is labelled) for binding to an antibody, which is a technique known to those skilled in the art.

As used herein, the term “target peptide” refers to a peptide comprising or consisting of the HNE-generated neo-epitope, consisting of an N-terminus or C-terminus sequence of the HNE-generated fragment of calprotectin, that is specifically recognised and bound by the monoclonal antibody.

As used herein the term “sandwich immunoassay” refers to an immunoassay that uses at least two antibodies for the detection of an antigen in a sample, and is a technique known to the person skilled in the art.

As used herein the term “ELISA” (enzyme-linked immunosorbent assay) refers to an immunoassay in which the target peptide present in a sample (if any) is detected using antibodies linked to an enzyme, such as horseradish peroxidase or alkaline phosphatase. The activity of the enzyme is then assessed by incubation with a substrate generating a measurable product. The presence and/or amount of target peptide in a sample can thereby be detected and/or quantified. ELISA is a technique known to those skilled in the art.

As used herein the term “amount of binding” refers to the quantification of binding between monoclonal antibody and target peptide, which said quantification is determined by comparing the measured values of target peptide in the biofluid samples against a calibration curve, wherein the calibration curve is produced using standard samples of known concentration of the target peptide. In the specific assay disclosed infra which measures in biofluids target peptides having an HNE-generated neo-epitope consisting of an N-terminus sequence of the HNE-generated fragment of calprotectin: KLGHPDTLNQGEFKELV (“NBH-222”) (SEQ ID NO: 1), the calibration curve is produced using standard samples of known concentration of a calibration peptide having the N-terminus amino acid sequence KLGHPDTLNQ (SEQ. ID No. 12) (and which may in particular consist of the amino acid sequence KLGHPDTLNQ (SEQ ID NO: 12)). The values measured in the biofluid samples are compared to the calibration curve to determine the actual quantity of target peptide in the sample.

As used herein the “predetermined cut-off value” means an amount of binding that is determined statistically to be indicative of a high likelihood of a disease (i.e. a disease characterized by or exhibiting inflammation, such as for example an inflammatory driven disease) or a particular status or severity thereof (such as an active disease status or disease prognosis) in a patient, in that a measured value of a target peptide in a patient sample that is at or above the statistical cut-off value corresponds to at least a 70% probability, preferably at least an 75% probability, more preferably at least an 80% probability, more preferably at least an 85% probability, more preferably at least a 90% probability, and most preferably at least a 95% probability of the presence of said disease or said particular status or severity thereof.

As used herein the term “values associated with normal healthy subjects and/or values associated with known disease status or severity” means standardised quantities of the target peptide determined by the immunoassay method for subjects considered to be healthy, i.e. without a disease (i.e. a disease characterized by or exhibiting inflammation, such as for example an inflammatory driven disease), and/or standardised quantities of the target peptide determined by the immunoassay method for subjects known to have a disease (i.e. a disease characterized by or exhibiting inflammation, such as for example an inflammatory driven disease) of a known status or severity.

### FIGURES

FIG. 1: Overview of the sequence of the calprotectin proteins S100A9 and S100A8 (A and B), and the fragments thereof generated by human neutrophil elastase (HNE). The HNE cleavage points are depicted by a down arrow (↓), with the some of the resulting HNE-generated fragments being highlighted in grey, and with the N-terminus sequences that form all or the start of the N-terminus neo-epitopes of the fragments designated as NBH-222, NBH-223, NBH-224, NBH-225 and NBH-226 being highlighted in a darker shade of grey.

FIG. 2: Relative abundance of CPa9-HNE (the N-terminus neo-epitope of NBH-222) in samples containing HNE cleaved calprotectin, full length calprotectin or HNE, as tested by mass spec (A); the reactivity of the CPa9-HNE antibody and assay, as tested against the selection peptide, elongated peptide, truncated peptide and non-sense peptide (B); and the abundance of CPa9-HNE, as detected by the CPa9-HNE antibody and assay, in samples containing HNE cleaved calprotectin, full length calprotectin or HNE (C).

FIG. 3: Spearman rho correlation of CPa9-HNE to faecal calprotectin (faecal-CP) and neutrophil count (A and B).

FIG. 4: Measured levels of CPa9-HNE in serum from inflammatory bowel disease (IBD), ulcerative colitis (UC) and Crohn's disease (CD) patients and healthy subjects (A and C), and associated ROC curves (B, D and E). Data are depicted as mean with standard error of the mean (SEM). Asterisks (*) depicts significant differences: *P<0.05, **P<0.01, ***P<0.001, ****P<0.0001

FIG. 5: Correlation of CPa9-HNE and faecal calprotectin (faecal-CP) to endoscopic scores for ulcerative colitis (MES) and Crohn's disease (SES-CD) (A-D). R=correlation coefficient, P=P-value, SES-CD=Simple endoscopic score for Crohn's disease, MES=Mayo endoscopic score.

FIG. 6: Measured levels of CPa9-HNE in healthy subjects, UC patients in clinical remission, and UC patents with active disease and associated ROC curves (A-C); measured levels of faecal-CP in UC patients in clinical remission, and UC patents with active disease and associated ROC curves (D and E); and correlation of CPa9-HNE with the partial mayo score (pMayo) and Trulove and Witts (TW-score) (F and G).

FIG. 7: (A) Serum biomarker levels of CPa9-HNE in COPD patients and healthy controls. Data is shown as Tukey's boxplot with lower limit measurement range (LLMR) indicated as a dashed line. Significance was found with a two-tailed Mann-Whitney test. (B) A ROC curve showing the diagnostic ability of CPa9-HNE in COPD patients. AUC: 0.9996.

FIG. 8: (A) Serum biomarker levels of CPa9-HNE in IPF patients and heathy controls. Data is shown as Tukey's boxplot with LLMR indicated as a dashed line. Significance was found with a two-tailed Mann-Whitney test. (B) A ROC curve showing the diagnostic ability of CPa9-HNE in IPF patients. AUC: 0.9813.

FIG. 9: Kaplan Meier plot for evaluating progression-free survival and overall survival in patients with metastatic melanoma treated with PD-1 inhibitor associated with CPa9-HNE at baseline by grouping (dichotomizing) at the 75th percentile (Q1+Q2+Q3 vs Q4).

FIG. 10: Serum biomarker levels of CPa9-HNE in lung cancer patients and controls. Data is shown as a scatter dot plot with line at median, and lower limit measurement range (LLMR) indicated as a dashed line. Significance was found by initially testing for normality and lognormality, where after a Dunnett's multiple comparisons test was applied.

FIG. 11: Serum biomarker levels of CPa9-HNE in joint disease patients and heathy controls. Data is shown as Tukey's boxplot.

### EXAMPLES

The presently disclosed embodiments described in the following examples are set forth to aid in the understanding of the disclosure, and should not be construed to limit in any way the scope of the disclosure as defined in the claims which follow thereafter. The following examples are put forth so as to provide those of ordinary skill in the art with a complete disclosure and description of how to make and use the described embodiments, and are not intended to limit the scope of the present disclosure nor are they intended to represent that the experiments below are all or the only experiments performed. Efforts have been made to ensure accuracy with respect to numbers used (e.g. amounts, temperature, etc.) but some experimental errors and deviations should be accounted for. Unless indicated otherwise, parts are parts by weight, molecular weight is weight average molecular weight, temperature is in degrees Centigrade, and pressure is at or near atmospheric.

In the following examples, the following materials and methods were employed.

### Methods

### In Vitro Cleavage of Calprotectin

Calprotectin fragments were generated in vitro by adding purified calprotectin, a heterodimer composed of the proteins S100A9 and S100A8, to an Eppendorf tube and adding human neutrophil elastase (HNE) in a protein:protease ratio of 100:1 (10 ug of calprotectin per 0.1 ug of HNE). The proteolytic reaction was inhibited after 24 hours by adding 5 mM EDTA stop buffer. Eppendorf tubes containing only protease buffer, or calprotectin without HNE, or HNE without calprotectin served as experimental controls.

### Mass Spectrometry Analysis

100 μl of cleaved samples or controls were desalted with reverse-phase Vydac UltraMicro Spin C18 columns (Harvard Apparatus, cat #74-7206) according to the manufacturer's instructions. Non-targeted mass spectrometry analysis was performed on a quadrupole Orbitrap benchtop mass spectrometer, QExactive, (Thermo Scientific) equipped with an Easy nano-LC 1000 system (ThermoFisher Scientific). Separation was performed on 75 μm×25 cm, Acclaim Pepmap™ RSLC C18 capillary columns packed with 2 μm particles (ThermoFisher Scientific). A spray voltage of +2000 V was used with a heated ion transfer setting of 275° C. for desolvation. The on-line reversed-phase separation was performed using a flow rate of 300 nl/min and a linear binary gradient 85 min was used. The gradient started with 3% solvent B for 4 min, then going to 35% solvent B in 64 min, after which it goes to 45% solvent B in 5 min. Finally, the organic solvent concentration was increased up to 90% in 5 min and kept at 90% for 7 min. An MS scan (400-1200 m/z) was recorded in the Orbitrap mass analyzer set at a resolution of 70,000 at 200 m/z, 1×10⁶ automatic gain control (AGC) target and 100 ms maximum ion injection time (44). The MS was followed by data-dependent collision-induced dissociation MS/MS scans at a resolution of 17,500 on the 15 most intense multiply charged ions at 2×10⁴ intensity threshold, 2 m/z isolation width and dynamic exclusion enabled for 30 s.

### Calprotectin Fragment Identification

Identification from discovery data was performed using the Homo sapiens proteome (UniProt proteome ID UP000005640, n20200 downloaded Dec. 6, 2015 with Proteome Discoverer 2.1 software (ThermoFisher Scientific). The processing workflow consisted of the following nodes: Spectrum Selector for spectra pre-processing (precursor mass range: 100-10000 Da; S/N Threshold: 1.5), Sequest-HT search engine (Protein Database: see above; Enzyme: No Enzyme; Max. missed cleavage sites: 2; Peptide length range 6-144 amino acids; Precursor mass tolerance: 10 ppm; Fragment mass tolerance: 0.02 Da; Dynamic modification: oxidation; Static modification: cysteine carbamidomethylation; and Percolator for peptide validation (FDR<0.01 based on peptide q-value). Peptide intensities were quantified using a proprietary algorithm developed in Proteome Discoverer 2.1 (ThermoFisher Scientific).

### Monoclonal Antibody Production and Clone Characterization

Briefly, generation of monoclonal antibodies targeting an HNE-generated neo-epitope consisting of an N-terminus or C-terminus sequence of an HNE-generated fragment of calprotectin (more specifically, targeting the N-terminus neo-epitope, also referred to herein as “CPa9-HNE”, of the HNE-generated fragment of calprotectin referred to as “NBH-222”) was carried out as follows.

Four to six-week old Balb/C mice were immunized subcutaneously with 200 μL emulsified antigen and 50 μg immunogenic peptide (KLGHPDTLNQ-GGC-Keyhole Limpet Hemocyanin (KLH) (SEQ ID NO: 24)) using Freund's incomplete adjuvant (Sigma-Aldrich). The mice were immunized at two-week intervals until stable serum titer levels were reached. The mouse with the highest serum titer was selected for monoclonal antibody production. The mouse was rested for one month and was then immunized intravenously with 50 μg immunogenic peptide in 100 μL 0.9% sodium chloride (NaCl) solution. After 3 days, splenocytes were isolated for cell fusion. In brief, splenocytes were fused with SP2/0 myeloma cells to produce hybridoma cells and then cloned in culture dishes using the semi-medium method. The clones were plated into 96-well microtiter plates, and limited dilution was used to secure monoclonal growth. The supernatants were screened for reactivity against the selection peptide (KLGHPDTLNQ (SEQ ID NO: 12)) and native material (serum and cleavage material) in an indirect competitive ELISA using streptavidin-coated plates (Roche, Hvidovre, Denmark, cat. 11940279). The clones with the best reactivity were purified using protein-G-columns according to the manufacturer's instructions (GE healthcare Life Sciences, Little Chalfont, Buckinghamshire, UK). These clones were tested for their reactivity toward the selection peptide and the elongated peptide, truncated peptide and non-sense peptide (see Table 1), and the clone showing the highest selectivity towards the selection peptide was chosen for monoclonal antibody production and assay development. Optimal incubation buffer, time, temperature, and optimal ratio between the biotinylated peptide and antibody were determined.

TABLE 1
overview of peptides

| | |
|---|---|
| Selection sequence | KLGHPDTLNQ (SEQ ID NO: 12) |
| Elongated sequence | VKLGHPDTLNQ (SEQ ID NO: 8) |
| Truncated sequence | LGHPDTLNQ (SEQ ID NO: 9) |
| non-sense peptide | YRDDLKKLLET (SEQ ID NO: 6) |
| (sequence of the | |
| NBH-225 fragment of | |
| calprotectin | |
| fragment was used) | |

The monoclonal antibody chosen for production and assay development was also sequenced, and the CDRs and isotype determined. The sequence of the chains are as follows (CDRs underlined and in bold; N-terminus signal peptide and C-terminus Constant region in italics):

Heavy Chain Sequence (Mouse IgG1 isotype)
(SEQ ID NO: 25)
MEWRIFLFILSGTAGVHSQVQLQQSGPELVKPGASVKMSCKASGYTFTD
HVINWVRQRTGQGLEWIGEIYPGSGSTYYNEKFKGKATLTADKSSNTAY
MQLSSLTSEDSAVYFCAWFAYWGQGTLVTVSAAKTTPPSVYPLAPGSAA
QTNSMVTLGCLVKGYFPEPVTVTWNSGSLSSGVHTFPAVLQSDLYTLSS
SVTVPSSTWPSETVTCNVAHPASSTKVDKKIVPRDCGCKPCICTVPEVS
SVFIFPPKPKDVLTITLTPKVTCVVVDISKDDPEVQFSWFVDDVEVHTA
QTQPREEQFNSTFRSVSELPIMHQDWLNGKEFKCRVNSAAFPAPIEKTI
SKTKGRPKAPQVYTIPPPKEQMAKDKVSLTCMITDFFPEDITVEWQWNG
QPAENYKNTQPIMDTDGSYFVYSKLNVQKSNWEAGNTFTCSVLHEGLHN
HHTEKSLSHSPGK
Light Chain Sequence (Mouse Kappa Isotype)
(SEQ ID NO: 26)
MESQTQVLISLLFWVSGACGDIVMTQSPSSLSVSAGEKVTMSCKSSQSL
LNSGNQKNYLAWYQQKPGQPPKLLIYGASTRESGVPDRFTGSGSGTDFT
LTISSVQAEDLAVYYCLNDHSYPYTFGGGTKLEIKRADAAPTVSIFPPS
SEQLTSGGASVVCFLNNFYPKDINVKWKIDGSERQNGVLNSWTDQDSKD
STYSMSSTLTLTKDEYERHNSYTCEATHKTSTSPIVKSFNRNEC

### Immunoassay (ELISA) Protocol

The levels of an HNE-generated neo-epitope consisting of an N-terminus or C-terminus sequence of an HNE-generated fragment of calprotectin (more specifically the levels of CPa9-HNE, i.e. the N-terminus neo-epitope of the HNE-generated fragment of calprotectin NBH-222) were assessed in samples by a solid phase competitive enzyme linked immunosorbent assay, which was carried out as follows.

96-well plates pre-coated with streptavidin (Roche Diagnostic's cat. No. 11940279, Hvidovre, Denmark) were coated with a biotinylated antigen (KLGHPDTLNQ-K-Biotin (SEQ ID NO: 27)) by incubation with the biotinylated antigen for 30 minutes at room temperature. Unbound biotinylated coater antigen was discarded, and the wells were washed with washing buffer (25 mM TRIZMA, 50 mM NaCl, 0.036% Bronidox L5, 0.1% Tween 20) using a standardized ELISA plate washing machine (BioTek® Instruments, Microplate washer, ELx405 Select CW, Winooski, USA). Samples were diluted in incubation buffer containing 1% bovine serum albumin (Sigma Aldrich, cat. No. a-7906, 98 purity) to maintain protein stability and for blocking. Samples and controls were added to the wells and incubated with the primary monoclonal antibody against the HNE-generated neo-epitope (CPa9-HNE), at 20° C. for 1 hour and agitated at 300 rpm. Unbound primary antibody and sample were discarded, and the wells were washed with washing buffer. Subsequently, HRP conjugated AffiniPure Rabbit anti-mouse IgG secondary antibodies (Jackson cat. No 315-035-045) was added to the wells and incubated for 1 hour at 20° C. Unbound secondary antibody was discarded by washing the wells in washing buffer. Chemiluminescence substrate (Roche Diagnostic's cat. No. 11582950001) was added to the well (100 μl/well), and the plates were incubated for 3 minutes at room temperature before reading the plates. Finally, an ELISA reader (VersaMAX; Molecular Devices, Wokingham Berkshire, UK) was used to quantify the relative light units (RLU) emitted from the plates at 440 nm and 650 nm. A standard curve was plotted using a 4-parametric mathematical fit model.

### Immunoassay Development

To test the robustness of the above assay, dilution recovery, peptide spiking in serum, analyte stability, freeze/thaw, antibody specificity (sanity test), interference (haemolysis, biotin, and lipids spiked in serum), inter/intra variance and biological relevance (cleavage material, serum and plasma) were tested.

### Biological Validation in IBD Patients

### Patient Demographics

Serums samples were tested from a patient cohort consisting of a total of 29 UC and 72 CD patients. Demographical data, disease history and therapy were obtained from electronic medical records and questionnaires. Anthropometric parameters were measured at the inclusion. Where available, patients' endoscopic disease activity was based on the simple endoscopic score for CD (SES-CD), and the Mayo endoscopic score for UC (MES). The MES score was used to add the information about disease extension²⁶. Inflammatory activity was also defined as combination of clinical and biochemical disease activity using Crohn's Disease Activity Index (CDAI), partial Mayo score (pMayo) and C-Reactive Protein (CRP). Patient stratification based on endoscopic scores was done as follows: SES-CD (remission=0-2, mild=3-6, moderate=7-15, severe>15), MES (remission=0-2, mild=3-6, moderate=7-15, severe>15). Clinical and biochemical activity was defined as CDAI≥150 or CRP>5 for CD and pMayo>1 or CRP>5 for UC. Disease severity and extension were assessed by Montreal classification.

### Statistical Analysis

To achieve normal distribution, log-transformation of the data was applied prior to statistical analysis. Student t-test and one-way ANOVA for normally distributed data was applied for analysing statistical differences. If a normal distribution was not achieved by log transformation Mann-Whitney U-test and Kruskal Wallis was applied. False discovery rate method (FDR=5%) was used for multiple comparisons' correction. Target peptide levels are presented as non-log transformed data with mean and standard error of the mean (SEM).

For the purpose of assessing the diagnostic power of the target peptide, receiver operating characteristic (ROC) curves were calculated. A P-value of ≤0.05 was considered statistically significant. Statistical analysis was performed using Graphpad Prism 7.03 and MedCalc. Figures were made using GraphPad Prism version 7.03.

### Biological Validation in COPD and IPF Patients

CPa9-HNE was measured in serum from COPD patients (n=68) and healthy controls (n=36), and IPF patients (n=16) and healthy controls (n=10).

### Biological Validation in Metastatic Melanoma Patients

CPa9-HNE was measured in pre-treatment serum from metastatic melanoma patients treated with anti-PD-1 therapy (pembrolizumab) (n=35). The patients were treated with pembrolizumab as the standard of care at Copenhagen University Hospital, Herlev, after informed consent and approval by the Ethics Committee for the Capital Region of Denmark in compliance with the Helsinki Declaration 1975. Serum samples were measured blinded. The association between CPa9-HNE levels and progression-free survival (PFS) and overall survival (OS) were assessed by Kaplan Meier analyses and Cox regression analyses, alone and after adjusting for PDL1 expression (≥1%), lactate dehydrogenase (LDH), BRAF mutational status, and C-reactive protein (CRP).

### Biological Validation in SCLC and NSCLC Patients

CPa9-HNE was measured in serum from patients with small cell lung cancer (SCLC), patients with non-small cell lung cancer (NSCLC), and healthy controls.

### Biological Validation in Joint Disease Patients

CPa9-HNE was measured in serum from patients with rheumatoid arthritis (n=15, age [range: 39-47]), psoriatic arthritis (n=11, age [range:31-64]), psoriasis (n=12, age [range: 27-52]), ankylosing spondylitis (n=11, age [range: 35-53]), young osteoarthritis (n=13, age<51 [range: 41-50]), old osteoarthritis (n=10, Age>50) and healthy controls (n=33). 41 of the patients were female.

### Results

### Calprotectin Neo-Epitope Generation and Identification

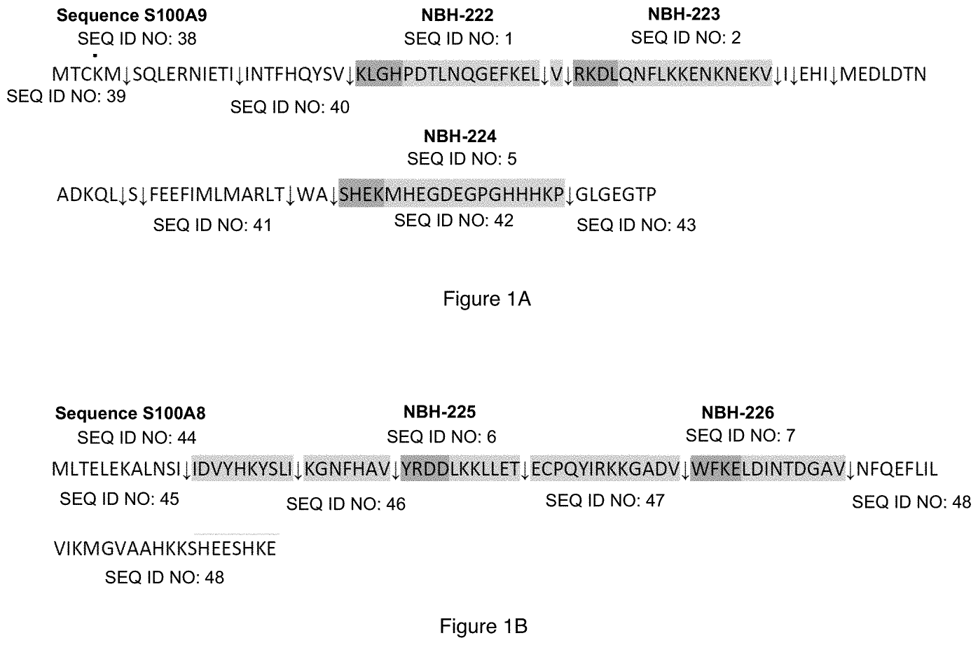

The non-targeted mass spectrometry analysis and subsequent calprotectin fragment identification, applying UniProt proteome ID UP000005640, revealed that several neo-epitope containing calprotectin fragments were generated by HNE, from both the S100A9 and S100A8 proteins (Table 2, and FIG. 1). Five HNE-generated calprotectin fragments (NBH-222, NBH-223, NBH-224, NBH-225, NBH-226) were considered as front runners for assay development, based on their PSM, Quality q-value and Quality PEP scores. Of these, due its PSM count (Table 2) and the uniqueness of its N-terminus neo-epitope sequence, NBH-222 was selected as the calprotectin (S100A9) fragment for development of an immunoassay targeting the HNE-generated N-terminus neo-epitope (CPa9-HNE) of this fragment.

TABLE 2
P06702 [Protein S100-A9 OS = Homo sapiens GN= S100A9 PE= 1 SV = 1]

| | | | | | | Percolator | Percolator |
|---|---|---|---|---|---|---|---|
| | | | Qvality | XCorr | Confidence | q- | PEP |
| Annotated | # | Qvality | q- | Sequest | Sequest | Value | Sequest |
| Sequence | PSMs | PEP | value | HT | HT | Sequest HT | HT |
| [V].KLGHPDTLNQ | 252 | 0.0763012 | 0 | 4.19 | High | 0 | 4.95E-05 |
| GEFKELV.[R] | | | | | | | |
| (SEQ ID NO: 1) | | | | | | | |
| [V].RKDLQNFLKKE | 216 | 0.0347011 | 0 | 5.44 | High | 0 | 1.58E-05 |
| NKNEKV.[I] | | | | | | | |
| (SEQ ID NO: 2) | | | | | | | |
| [V].RKDLQNFLKK | 197 | 0.0429293 | 0 | 5.43 | High | 0 | 0.001887 |
| ENKNEKVI.[E] | | | | | | | |
| (SEQ ID NO: 3) | | | | | | | |
| [I].EHIMEDLDTNAD | 189 | 0.108379 | 0 | 4.27 | High | 0 | 0.0001494 |
| KQL.[S] | | | | | | | |
| (SEQ ID NO: 4) | | | | | | | |
| [A].SHEKMHEGDE | 163 | 0.0821572 | 0 | 6.01 | High | 0 | 0.0005104 |
| GPGHHHKPGLGE | | | | | | | |
| GTP.[-] | | | | | | | |
| (SEQ ID NO: 5) | | | | | | | |
| [I].MEDLDTNADKQ | 111 | 0.212839 | 0 | 2.97 | High | 0 | 0.0005214 |
| L.[S] | | | | | | | |
| (SEQ ID NO: 28) | | | | | | | |
| [I].EHIMEDLDTNAD | 63 | 0.136895 | 0 | 2.88 | High | 0 | 0.006693 |
| KQLS.[F] (SEQ ID | | | | | | | |
| NO: 29) | | | | | | | |
| [M].SQLERNIETI.[I] | 48 | 0.294959 | 0 | 1.92 | High | 0.003407 | 0.08593 |
| (SEQ ID NO: 30) | | | | | | | |
| [V].KLGHPDTLNQG | 42 | 0.177644 | 0 | 4.18 | High | 0 | 0.0005776 |
| EFKEL.[V] | | | | | | | |
| (SEQ ID NO: 31) | | | | | | | |
| [S].HEKMHEGDEG | 32 | 0.0745309 | 0 | 5.06 | High | 0 | 0.0003257 |
| PGHHHKPGLGEGT | | | | | | | |
| PH | | | | | | | |
| (SEQ ID NO: 32) | | | | | | | |
| [T].WASHEKMHEG | 32 | 0.0649674 | 0 | 7.46 | High | 0 | 8.10E-05 |
| DEGPGHHHKPGL | | | | | | | |
| GEGTP.[-] | | | | | | | |
| (SEQ ID NO: 33) | | | | | | | |
| [M].HEGDEGPGHH | 30 | 0.252624 | 0 | 4.00 | High | 0 | 0.005602 |
| HKPGLGEGTP.[-] | | | | | | | |
| (SEQ ID NO: 34) | | | | | | | |
| [I].EHIMEDLDTNAD | 25 | 0.16537 | 0 | 3.14 | High | 0 | 0.0001334 |
| KQL.[S] | | | | | | | |
| (SEQ ID NO: 4) | | | | | | | |
| [I].MEDLDTNADKQ | 21 | 0.163632 | 0 | 2.56 | High | 0 | 0.0001257 |
| LS.[F] | | | | | | | |
| (SEQ ID NO: 35) | | | | | | | |
| [A].SHEKMHEGDE | 13 | 0.34117 | 0 | 4.13 | High | 0.001932 | 0.06063 |
| GPGHHHKPGLGE | | | | | | | |
| GTP.[-] | | | | | | | |
| (SEQ ID NO: 5) | | | | | | | |
| [I].MEDLDTNADKQ | 12 | 0.263837 | 0 | 2.65 | High | 0 | 0.001686 |
| L.[S] | | | | | | | |
| (SEQ ID NO: 28) | | | | | | | |
| [A].SHEKMHEGDE | 11 | 0.222026 | 0 | 4.72 | High | 0 | 0.0008662 |
| GPGHHHKPG.[L] | | | | | | | |
| (SEQ ID NO: 36) | | | | | | | |

P05109 [Protein S100-A8 OS = Homo sapiens GN = S100A8 PE = 1 SV = 1]

| | | | | | | Percolator | Percolator |
|---|---|---|---|---|---|---|---|
| | | | Qvality | XCorr | Confidence | q- | PEP |
| Annotated | # | Qvality | q- | Sequest | Sequest | Value | Sequest |
| Sequence | PSMs | PEP | value | HT | HT | Sequest HT | HT |
| [V].YRDDLKKLLET | 112 | 0.301257 | 0 | 2.21 | High | 0.00481 | 0.1006 |
| [E] | | | | | | | |
| (SEQ ID NO: 6) | | | | | | | |
| [V].WFKELDINTDG | 39 | 0.32248 | 0 | 2.50 | High | 0 | 0.004899 |
| AV.[N] | | | | | | | |
| (SEQ ID NO: 7) | | | | | | | |
| [I].IDVYHKYSLI.[K] | 2 | 0.327439 | 0 | 1.92 | High | 0 | 0.005295 |
| (SEQ ID NO: 37) | | | | | | | |

### Immunoassay Development for CPa9-HNE

### Specificity, Accuracy and Precision

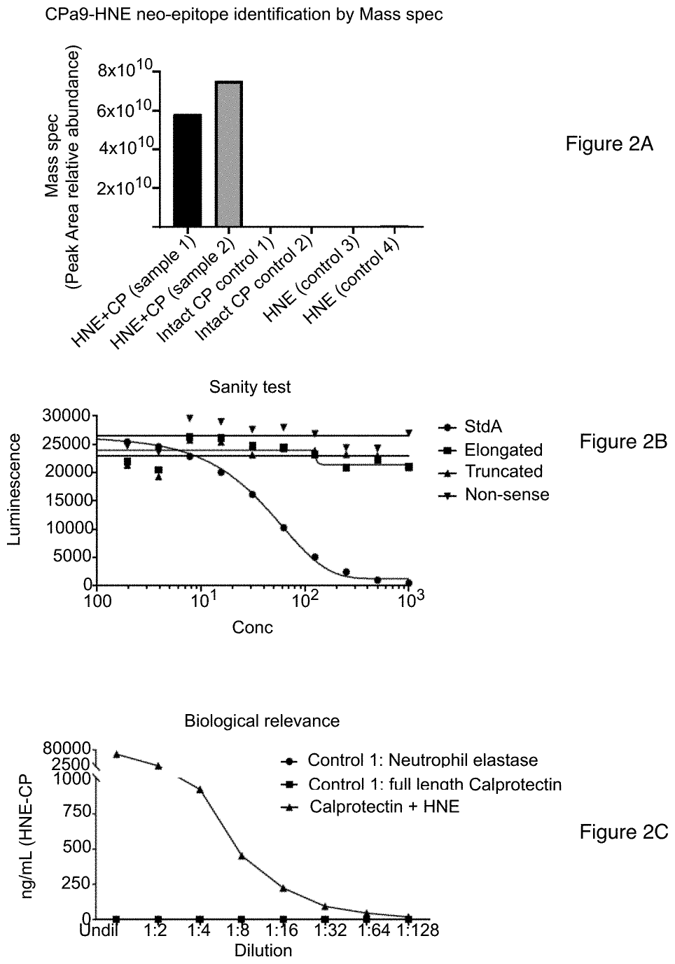

The mass spectrometry data demonstrated that NBH-222 was only present in samples containing full length human calprotectin+human neutrophil elastase, but not in the control samples containing only full-length human calprotectin or human neutrophil elastase (FIG. 2A). A monoclonal antibody (also referred to herein as the CPa9-HNE antibody) targeting CPa9-HNE (the HNE-generated N-terminus neo-epitope of NBH-222) was developed and used in an ELISA protocol as described above (also referred to hereinafter as the CPa9-HNE assay), and the specificity of this antibody when used in this immunoassay was tested against the selection peptide and the elongated peptide, truncated peptide and non-sense peptide (having the sequences set out in Table 1). The Cpa9-HNE antibody demonstrated only reactivity towards the Cpa9-HNE neo-epitope sequence (FIG. 2B). In samples containing HNE cleaved calprotectin, or only intact full-length human calprotectin, or only human neutrophil elastase, the CPa9-HNE antibody was able to identify the target neo-epitope sequence only when calprotectin cleaved by human neutrophil elastase was present (FIG. 2C). The final specifications of the CPa9-HNE assay are as set out in table 3.

TABLE 3
CPa9-HNE assay parameters

| Species possibilities | Human |
|---|---|
| Analyte stability, 4° C. | Minimum 48 hours |
| Analyte stability, 20° C. | 24 hours |
| intra-assay CV %, QC | 6% |
| Inter-assay CV %, QC | 7.5% |
| RnD Assay range | 9.08 ng/mL-492.7 ng/mL |
| (ng/ml)_{LLMR-ULMR} | |
| Std.A, conc. (ng/ml) | 1000 ng/mL |
| Mean relative light | 48251.074 |
| units (RLU) | |
| value, StdH (Abs) | |
| IC50 (mean, ng/ml) | 78.34 |
| Slope (mean) | 0.94 |
| CO1 (ng/ml); mean | 36.96 ng/mL |
| (± 20%) | (29.57-44.35 ng/mL) |
| CO2 (ng/ml); mean | 248.2 ng/mL |
| (± 20%) | (198.6-297.8 ng/mL) |

### Patient Demographics in IBD Patients

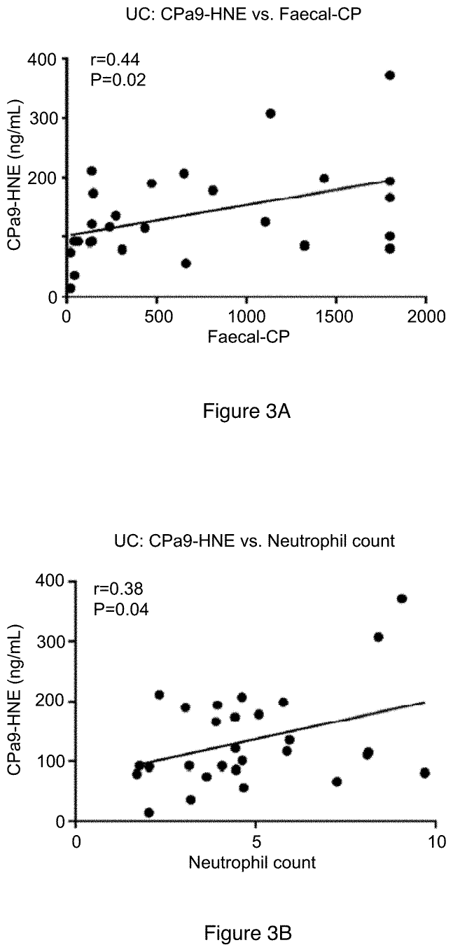

CPa9-HNE levels in serum from IBD patients were demonstrated to correlate to faecal calprotectin levels (i.e. levels of intact calprotectin in faeces) and neutrophil count in UC patients (FIGS. 3A and B).

### CPa9-HNE Serum Levels in IBD

### CPa9-HNE is Elevated in Serum of IBD Compared to Healthy Controls

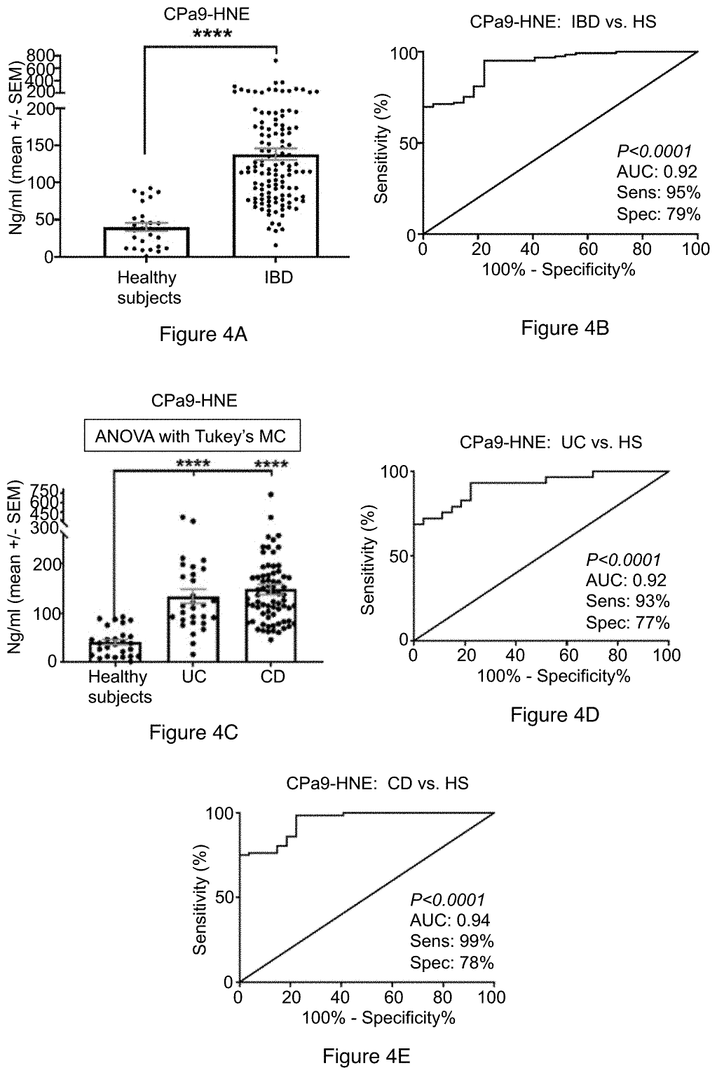

The serum levels of CPa9-HNE were measured in serum from UC, CD and healthy subjects. CPa9-HNE was demonstrated to be ˜4-fold higher in the serum of IBD patients compared healthy subjects (AUC: 0.92, P<0.0001) (FIGS. 4A and B). When dividing the patients into CD and UC, serum CPa9-HNE was equally elevated in UC and CD patients, and was ˜4-fold higher in CD (AUC: 0.92, P<0.0001) and UC (AUC: 0.94, P<0.0001) patients compared to healthy subjects (FIGS. 4C, D and E).

CPa9-HNE is Associated with Endoscopic Disease Activity in UC and CD

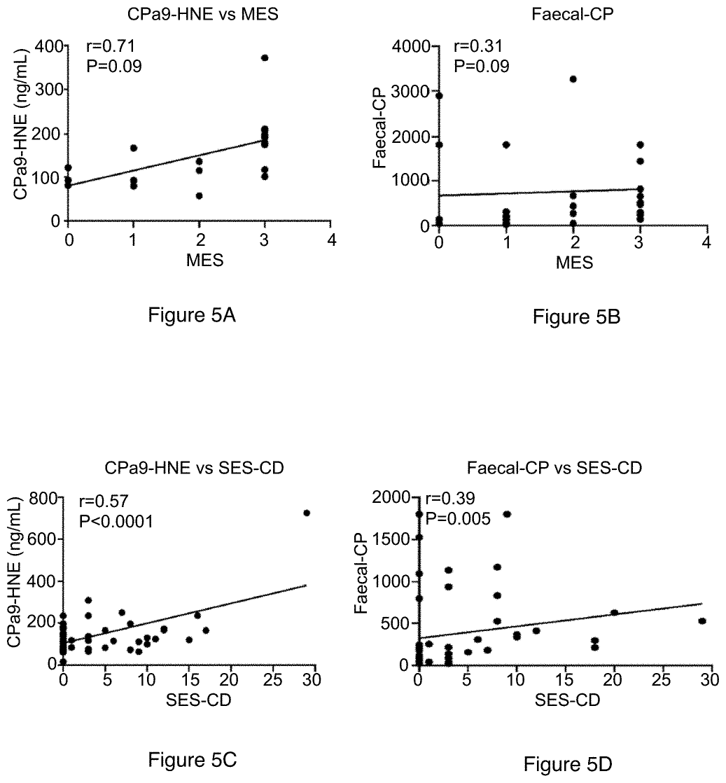

CPa9-HNE correlated well with endoscopic disease activity for CD (SES-CD: r=0.057, P<0.0001) and UC (MES: r=0.71, P=0.0003) (FIGS. 5A and B). Faecal-CP correlated with the endoscopic disease activity for CD (SES-CD: r=0.39, P=0.005) but not for UC (MES: r=0.31, P=0.09) (FIGS. 5C and D)

CPa9-HNE is Associated with Clinical Disease Activity in UC

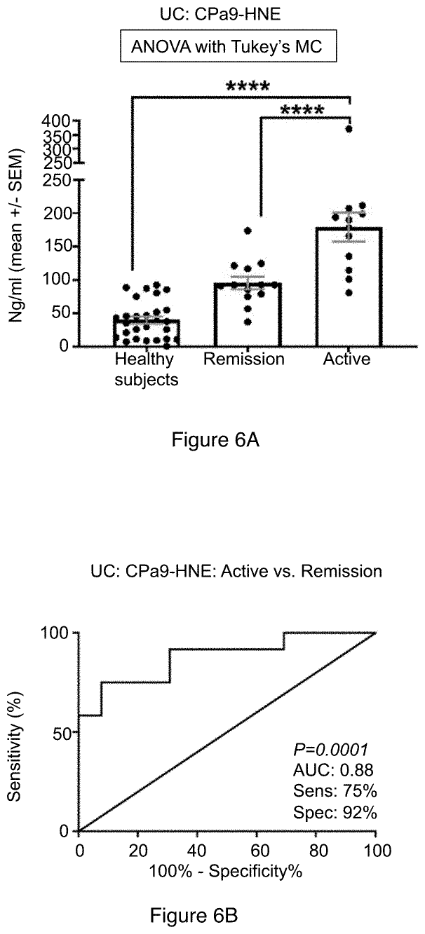

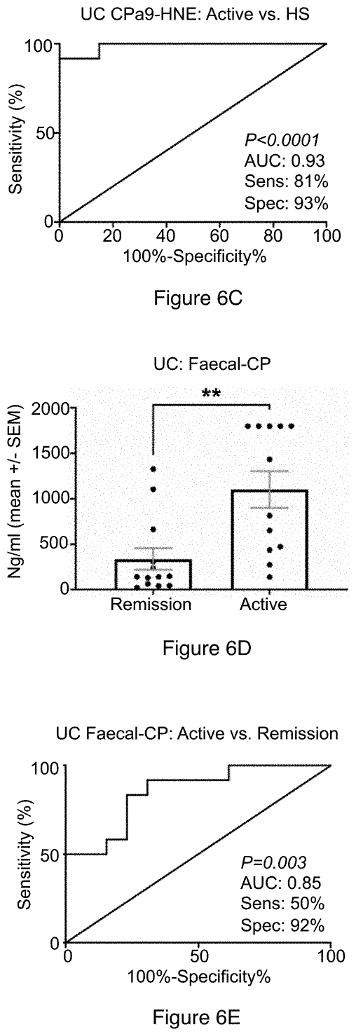

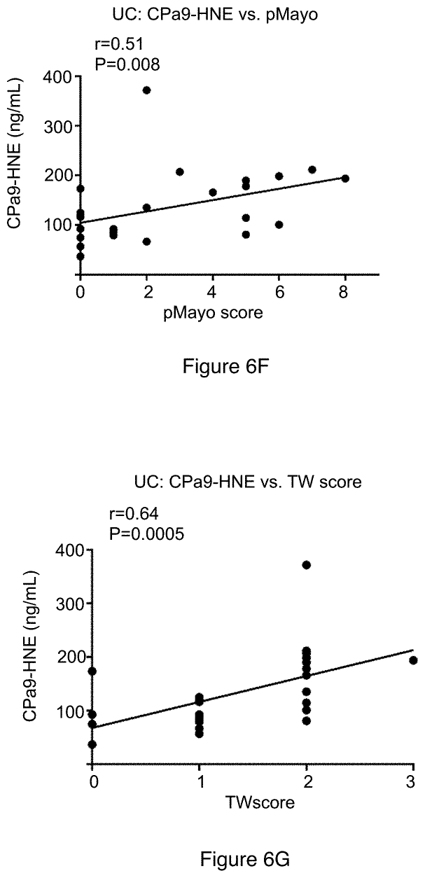

Dividing the UC patients into clinical remission and clinical active disease based on the partial mayo score, CPa9-HNE was significantly elevated in UC patients with clinical active disease compared to UC patients in remission (AUC: 0.88, P<0.0001) and healthy donors (AUC: 0.93, P<0.0001) (FIGS. 6A, B and C). The performance of CPa9-HNE in comparison to faecal calprotectin then CPa9-HNE demonstrated to be equally or slightly better than faecal calprotectin with increased AUC and sensitivity (FIGS. 6A to E). CPa9-HNE also demonstrated to correlate with the partial mayo score (r=0.51, P=0.008) and the Truelove and Witts score (r=0.64, P=0.0005) for UC (FIGS. 6F and G).

### CPa9-HNE is Elevated in COPD Patients Compared to Healthy Controls

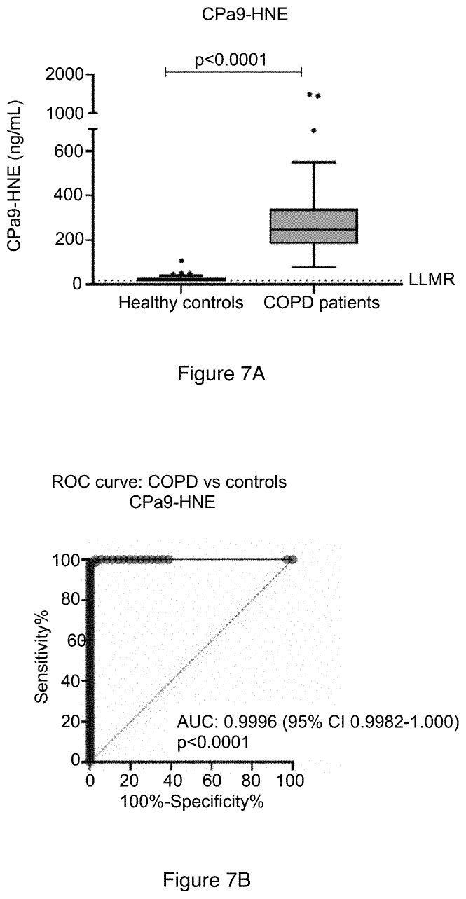

CPa9-HNE was measured in serum samples for COPD patients and healthy controls. FIG. 7A displays the difference of the invention between the healthy controls (n=36) and COPD patients (n=68), (p<0.0001). Furthermore, the diagnostic ability was calculated in a receiver operating characteristics (ROC) curve with an area under the curve (AUC) determined as 0.9996, FIG. 7B. The measurements were not affected by the patients' BMI, age, and sex.

### CPa9-HNE is Elevated in IPF Patients Compared to Healthy Controls

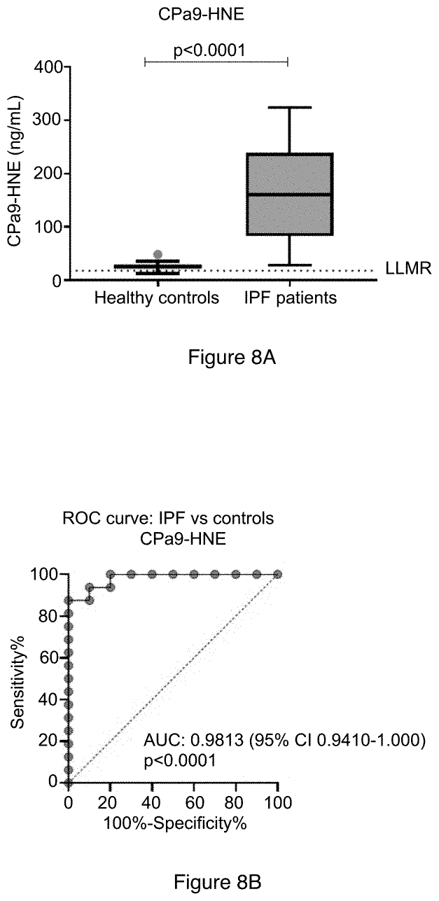

CPa9-HNE was measured in serum samples for IPF patients (n=16) and healthy controls (n=10). FIG. 8A displays the difference of the Invention between healthy controls and patients with IPF (p<0.0001). Moreover, the diagnostic ability was calculated in a ROC curve with an AUC determined as 0.9813, FIG. 8B.

High CPa9-HNE is Associated with a Worse Prognosis in Metastatic Melanoma

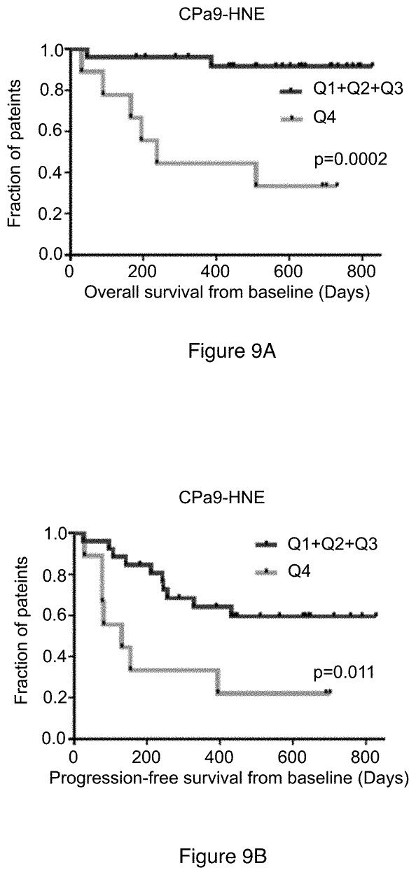

The association between CPa9-HNE and survival outcomes in the metastatic melanoma patients was evaluated by Kaplan-Meier analysis. Using the 75th percentile cut point, it was found that patients with high CPa9-HNE levels (>75th percentile) had significantly worse PFS (p=0.011) and OS (p=0.0002) compared to patients with low CPa9-HNE levels (FIG. 9). In support, univariate Cox regression identified high (>75th percentile) pre-treatment CPa9-HNE as predictor of worse PFS (HR=3.32, 95% C1=1.25-8.82, p=0.016) and OS (HR=11.31, 95% C1=2.27-56.33, p=0.003) when compared to low CPa9-HNE (Table 4). By multivariate Cox regression, high CPa9-HNE was found to be independently predictive of poor PFS (HR=8.22, 95% C1=1.30-52.14, p=0.025) and OS (HR=76.87, 95% C1=4.73-1248.57, p=0.002) when adjusted for PDL1 expression, LDH, BRAF mutations, and CRP (Table 4).

TABLE 4
Cox regression analysis for predicting progression-free survival and
overall survival outcome

| | Progression-free | | | | | |
|---|---|---|---|---|---|---|
| | survival | | | Overall survival | | |
| | HR | 95% Cl | p-value | HR | 95% Cl | p-value |
| Univariate analysis | | | | | | |
| CPa9-HNE, Q4 vs | 3.32 | 1.25-8.82 | 0.016 | 11.31 | 2.27-56.33 | 0.003 |
| Q1 + Q2 + Q3 | | | | | | |
| Multivariate analysis | | | | | | |
| adjusted for PDL1 | | | | | | |
| expression (≥1%) LDH, | | | | | | |
| BRAF mutations, and | | | | | | |
| CRP | | | | | | |
| CPa9-HNE, Q4 vs | 8.22 | 1.30-52.14 | 0.025 | 76.87 | 4.73-1248.57 | 0.002 |
| Q1 + Q2 + Q3 | | | | | | |

### CPa9-HNE is Elevated in SCLC and NSCLC Patients Compared to Healthy Controls

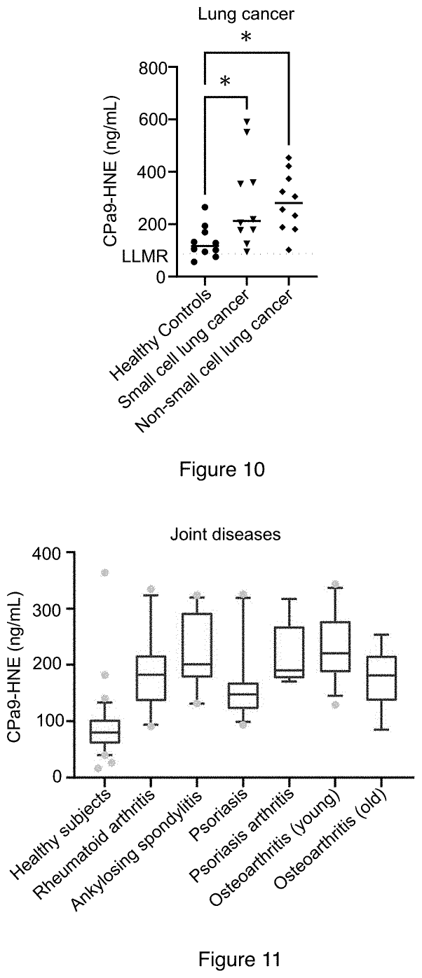

CPa9-HNE was measured in serum from patients with small cell lung cancer (SCLC), patients with non-small cell lung cancer (NSCLC), and healthy controls. The Lung cancer patients experienced a statistically significant increase in CPa9-HNE serum levels, where both small cell lung cancer (SCLC) (p=0.0435, n=10, median=212.1 [IQR 164.0-407.1]) and non-small cell lung cancer (NSCLC) (p=0.0467, n=10, median=281.3 [IQR 186.8-385.8]) were examined (FIG. 10).

### CPa9-HNE is Elevated in Joint Diseases Compared to Healthy Donors

The CPa9-HNE was measured in serum from patients with rheumatoid arthritis (n=15, age [range:39-47]), psoriatic arthritis (n=11, age [range:31-64]), psoriasis (n=12, age [range:27-52]), ankylosing spondylitis (n=11, age [range:35-53]), young osteoarthritis (n=13, age<51 [range:41-50]), old osteoarthritis (n=10, Age>50) and healthy controls (n=33). The CPa9-HNE biomarker was significantly elevated in all joint disease compared to healthy controls, with the greatest difference being observed for ankylosing spondylitis (p<0.001), psoriatic arthritis (p<0.001), young osteoarthritis (p<0.001), and rheumatoid arthritis (p<0.01) patients compared to healthy controls (FIG. 11).

### DISCUSSION

In this study the inventors have demonstrated that the CPa9-HNE assay is technically robust with biological and clinical relevance as a serum based assay for detecting, inter alia, IBD, COPD, IPF, SCLC, NSCLC, rheumatoid arthritis, ankylosing spondylitis, psoriasis, psoriasis arthritis and osteoarthritis, and assessing disease activity. Additionally, the inventors have demonstrated that the CPa9-HNE assay is of prognostic value in patients with metastatic melanoma.

As indicated in table 3 the CPa9-HNE containing calprotectin fragment (NBH-222) is stable at 4° C. for a minimum of 48 hours in serum, which also means that this neo-epitope containing fragment has a much longer half-life than intact calprotectin in serum and plasma, intact calprotectin having only a half-life of 5 hours in plasma (9) despite being proven to be stable in faeces for 7 days (1). This may explain the poor clinical applicability of the faecal calprotectin assays when applied to serum/plasma, which assays performance is similar to CRP (10-12). The high stability of the CPa9-HNE fragment in serum (minimum 48 hours at 4° celcius) may be explained by the fact that it is a degradation product/metabolite of calprotectin, making the fragment more resistant for further degradation. In addition, the CPa9-HNE assay is also more sensitive than the faecal calprotectin assay, since it measures nanograms/mL instead of micrograms/gram (27).

Calprotectin's ability to bind metal ions e.g. calcium and zinc ions makes it highly resistant towards to metalloproteinase (MMP) degradation since MMPs require Zinc ions for activation. HNE is a serine protease and does not require activation by metal ions and is thus able to degrade calprotectin and generate HNE derived neo-epitope containing calprotectin fragments (5, 28-30). Therefore, CPa9-HNE levels may be representative of local tissue inflammation, as CPa9-HNE containing fragments will only be generated by activated neutrophil granulocytes and other activated leukocytes, and only the presence of HNE. This, is in contrast to intact calprotectin which can be released randomly into the circulation and faeces by non-activated circulating leukocytes and to some extent by epithelial cells, which also express calprotectin (10,31,32).

CPa9-HNE was demonstrated to be highly abundant in serum of CD and UC patients, indicating high potential as surrogate biomarker aiding diagnosis of IBD. This is in concordance with faecal calprotectin as this biomarker can reliably distinguish between IBS and IBD patients (6-8). Furthermore, CPa9-HNE also correlated with faecal calprotectin, neutrophil granulocyte count and disease activity for CD and UC, indicating that CPa9-HNE can be used to monitor disease activity and may be surrogate biomarker for endoscopic assessment for CD and UC.

The CPa9-HNE assay also elucidated a significant difference between patients affected with pulmonary diseases COPD, and IPF as compared to healthy controls. The CPa9-HNE assay measures a specific cleavage site on calprotectin that is generated by neutrophil elastase. Therefore, these results indicate a higher activity of NE in these pulmonary diseases.

The CPa9-HNE assay was also demonstrated to be predictive of overall and progression free survival rates in patients with metastatic melanoma treated with treated with anti-PD-1 therapy, based on baseline (pre-treatment) concentrations of serum CPa9-HNE.

Finally, CPa9-HNE was also shown to be significantly elevated in the serum of lung cancer patients (both SCLC and NSCLC) as compared to healthy controls, and in the serum of rheumatoid arthritis, ankylosing spondylitis, psoriasis, psoriasis arthritis and osteoarthritis patients as compared to healthy controls.

### CONCLUSION

The CPa9-HNE ELISA was proven to have high specificity towards the neo-epitope both in in vitro cleavage samples and in the human IBD serum samples as a novel serum biomarker for HNE mediated degradation of calprotectin. CPa9-HNE was highly associated with CD and UC patients and demonstrated high diagnostic accuracy to differentiate IBD patients from healthy donors. Furthermore, CPa9-HNE also correlated with endoscopic disease activity for CD and UC, SES-CD and MES respectively. CPa9-HNE also correlated with clinical disease activity scores for UC (partial Mayo score and the Trulove and Witts score). Therefore, the CPa9-HNE is a surrogate biomarker of disease activity for CD and UC, and performed at least as well as or slightly better than Faecal-CP. Therefore, CPa9-HNE is a novel clinically relevant biomarker for IBD diagnosis and monitoring of disease activity, as well as also other inflammatory driven diseases including, rheumatoid arthritis, psoriasis, psoriasis arthritis, ankylosing spondylitis, osteoarthritis, Sjögrens syndrome, and lupus. It may also be uses to predict or monitor treatment efficacy in e.g. TNF-alpha, vedolizumab and ustekinumab prospective studies.

Additionally, CPa9-HNE has been shown to be a clinically relevant biomarker in lung diseases such as COPD and IPF, metastatic diseases such as metastatic melanoma, and lung cancers such as SCLC and NSCLC.

In this specification, unless expressly otherwise indicated, the word ‘or’ is used in the sense of an operator that returns a true value when either or both of the stated conditions is met, as opposed to the operator ‘exclusive or’ which requires that only one of the conditions is met. The word ‘comprising’ as used herein means ‘including’ or ‘consisting of’. All prior teachings acknowledged herein are hereby incorporated by reference.

### REFERENCES

1. Røseth AG., Fagerhol M K., Aadland E., et al. Assessment of the neutrophil dominating protein calprotectin in feces. A methodologic study. Scand J Gastroenterol 1992; 27(9):793-8. Doi: 10.3109/00365529209011186.
2. Stephan J R., Nolan E M. Calcium-induced tetramerization and zinc chelation shield human calprotectin from degradation by host and bacterial extracellular proteases. Chem Sci 2016; 7(3):1962-75. Doi: 10.1039/c5sc03287c.
3. Levine A P., Segal A W. What Is wrong with granulocytes in inflammatory bowel diseases. Dig Dis 2013; 31:321-7. Doi: 10.1159/000354686.
4. Geboes K. Histopathology of Crohn's Disease and Ulcerative Colitis. Inflammatory Bowel Disease, vol. 18. 2003. p. 255-76.
5. Wéra O., Lancellotti P., Oury C. The Dual Role of Neutrophils in Inflammatory Bowel Diseases. J Clin Med 2016; 5(12):118. Doi: 10.3390/jcm5120118.
6. Gionchetti P., Dignass A., Danese S., et al. 3rd European evidence-based consensus on the diagnosis and management of Crohn's disease 2016: Part 2: Surgical management and special situations. J Crohn's Colitis 2017; 11(2):135-49. Doi: 10.1093/ecco-jcc/jjw169.
7. Schoepfer A M., Trummler M., Seeholzer P., et al. Discriminating IBD from IBS: comparison of the test performance of fecal markers, blood leukocytes, CRP, and IBD antibodies. Inflamm Bowel Dis 2008; 14(1):32-9. Doi: 10.1002/ibd.20275.
8. Chang M-H., Chou J-W., Chen S-M., et al. Faecal calprotectin as a novel biomarker for differentiating between inflammatory bowel disease and irritable bowel syndrome. Mol Med Rep 2014; 10(1):522-6. Doi: 10.3892/mmr.2014.2180.
9. Hammer H B., Ødeg̊ard S., Fagerhol M K., et al. Calprotectin (a major leucocyte protein) is strongly and independently correlated with joint inflammation and damage in rheumatoid arthritis. Ann Rheum Dis 2007; 66(8):1093-7. Doi: 10.1136/ard.2006.064741.
10. Kopi T A., Shahrokh S., Mirzaei A., et al. The role of serum calprotectin as a novel biomarker in inflammatory bowel diseases: A review study. Gastroenterol Hepatol from Bed to Bench 2019; 12(3):183-9. Doi: 10.22037/ghfbb.v12i3.1591.
11. Meuwis M A., Vernier-Massouille G., Grimaud J C., et al. Serum calprotectin as a biomarker for Crohn's disease. J Crohn's Colitis 2013; 7(12):e678-83. Doi: 10.1016/j.crohns.2013.06.008.
12. McCann R K., Smith K., Gaya D R. A prospective single centre pilot evaluation of a serum calprotectin assay in unselected GI patients. Clin Biochem 2017; 50(9):533-6. Doi: 10.1016/j.clinbiochem.2017.01.006.
13. Carlsen K., Malham M., Hansen L F., et al. Serum Calprotectin in Adolescents With Inflammatory Bowel Disease—A Pilot Investigation. J Pediatr Gastroenterol Nutr 2019; 68(5).
14. Malham M., Carlsen K., Riis L., et al. Plasma calprotectin is superior to serum calprotectin as a biomarker of intestinal inflammation in ulcerative Colitis. Scand J Gastroenterol 2019; 54(10):1214-9. Doi: 10.1080/00365521.2019.1665097.
15. Kalla R., Kennedy N A., Ventham N T., et al. Serum Calprotectin: A Novel Diagnostic and Prognostic Marker in Inflammatory Bowel Diseases. Am J Gastroenterol 2016; 111(12).
16. Kirov S., Sasson A., Zhang C., et al. Degradation of the extracellular matrix is part of the pathology of ulcerative colitis. Mol Omi 2019; 15(1):67-76. Doi: 10.1039/c8mo00239h.
17. Urban C F., Ermert D., Schmid M., et al. Neutrophil Extracellular Traps Contain Calprotectin, a Cytosolic Protein Complex Involved in Host Defense against Candida albicans 2009; 5(10). Doi: 10.1371/journal.ppat.1000639.
18. Mortensen J H., Manon-Jensen T., Jensen M D., et al. Ulcerative colitis, Crohn's disease, and irritable bowel syndrome have different profiles of extracellular matrix turnover, which also reflects disease activity in Crohn's disease. PLoS One 2017; 12(10):1-16. Doi: 10.1371/journal.pone.0185855.
19. Kristensen J H., Karsdal M A., Sand J M., et al. Serological assessment of neutrophil elastase activity on elastin during lung ECM remodeling. BMC Pulm Med 2015; 15(1):53. Doi: 10.1186/s12890-015-0048-5.
20. Magro F., Gionchetti P., Eliakim R., et al. Third European Evidence-based Consensus on Diagnosis and Management of Ulcerative Colitis Part 1: Definitions, Diagnosis, Extra-intestinal Manifestations, Pregnancy, Cancer, Surveillance, Surgery, and Ileo-anal Pouch Disorders. J Crohn's Colitis 2017:1-115. Doi: 10.1093/ecco-jcc/jjx008.
21. Mortensen J H., Godskesen L E., Jensen M D., et al. Fragments of Citrullinated and MMP-degraded Vimentin and MMP-degraded Type III Collagen Are Novel Serological Biomarkers to Differentiate Crohn's Disease from Ulcerative Colitis. J Crohn's Colitis 2015:jjv123. Doi: 10.1093/ecco-jcc/jjv123.
22. van Haaften W T., Mortensen J H., Karsdal M A., et al. Misbalance in type III collagen formation/degradation as a novel serological biomarker for penetrating (Montreal B3) Crohn's disease. Aliment Pharmacol Ther 2017; (August 2016). Doi: 10.1111/apt.14092.
23. Goffin L., Fagagnini S., Vicari A., et al. Anti-MMP-9 Antibody: A Promising Therapeutic Strategy for Treatment of Inflammatory Bowel Disease Complications with Fibrosis. Inflamm Bowel Dis 2016; 22(9):2041-57. Doi: 10.1097/MIB.0000000000000863.
24. Lindholm M., Manon-Jensen T., Madsen G I., et al. Extracellular Matrix Fragments of the Basement Membrane and the Interstitial Matrix Are Serological Markers of Intestinal Tissue Remodeling and Disease Activity in Dextran Sulfate Sodium Colitis. Dig Dis Sci 2019. Doi: 10.1007/s10620-019-05676-6.
25. Mortensen J H., Lindholm M., Langholm L L., et al. The intestinal tissue homeostasis—the role of extracellular matrix remodeling in inflammatory bowel disease. Expert Rev Gastroenterol Hepatol 2019; 00(00):1-17. Doi: 10.1080/17474124.2019.1673729.
26. Lobatón T., Bessissow T., De Hertogh G., et al. The Modified Mayo Endoscopic Score (MMES): A New Index for the Assessment of Extension and Severity of Endoscopic Activity in Ulcerative Colitis Patients. J Crohns Colitis 2015; 9(10):846-52. Doi: 10.1093/ecco-jcc/jjv111.
27. Labaere D., Smismans A., Van Olmen A., et al. Comparison of six different calprotectin assays for the assessment of inflammatory bowel disease. United Eur Gastroenterol J 2014; 2(1):30-7. Doi: 10.1177/2050640613518201.
28. Döring G. The Role of Neutrophil Elastase in Chronic Inflammation. Am J Respir Crit Care Med 1994; 150(6_pt_2):5114-7. Doi: 10.1164/ajrccm/150.6_Pt_2.5114.
29. Alfakry H., Malle E., Koyani C N., et al. Neutrophil proteolytic activation cascades: a possible mechanistic link between chronic periodontitis and coronary heart disease. Innate Immun 2016; 22(1):85-99. Doi: 10.1177/1753425915617521.
30. Gouni-Berthold I. Neutrophil-elastase in chronic inflammatory bowel disease: A marker of disease activity? Hepatogastroenterology 1999; 46(28):2315-20.
31. Fukunaga S., Kuwaki K., Mitsuyama K., et al. Detection of calprotectin in inflammatory bowel disease: Fecal and serum levels and immunohistochemical localization. Int J Mol Med 2017:107-18. Doi: 10.3892/ijmm.2017.3244.
32. Tardif M R., Chapeton-Montes J A., Posvandzic A., et al. Secretion of S100A8, S100A9, and S100A12 by Neutrophils Involves Reactive Oxygen Species and Potassium Efflux. J Immunol Res 2015; 2015. Doi: 10.1155/2015/296149.
33. Kabat, E. A., T. T. Wu, H. M. Perry, K. S. Gottesman and C. Foeller (1987), Sequences of Proteins of Immunological Interest, United States Department of Health and Human Services, Bethesda, Md., p. 1
34. Martinez F J, Collard H R, Pardo A, Raghu G, Richeldi L, Selman M, Swigris J J, Taniguchi H, Wells A U. Idiopathic pulmonary fibrosis. Nat Rev Dis Primers. 2017 Oct. 20; 3:17074. doi: 10.1038/nrdp.2017.74. PMID: 29052582.
35. Sand J M, Martinez G, Midjord A K, Karsdal M A, Leeming D J, Lange P. Characterization of serological neo-epitope biomarkers reflecting collagen remodeling in clinically stable chronic obstructive pulmonary disease. Clin Biochem. 2016 October; 49(15):1144-1151. doi: 10.1016/j.clinbiochem. 2016.09.003. Epub 2016 Sep. 7. PMID: 27614218.
36. Jasper A E, McIver W J, Sapey E, Walton G M. Understanding the role of neutrophils in chronic inflammatory airway disease. F1000Res. 2019; 8:F1000 Faculty Rev-557. Published 2019 Apr. 26. doi:10.12688/f1000research.18411.1
37. Obayashi Y, Yamadori I, Fujita J, Yoshinouchi T, Ueda N, Takahara J. The role of neutrophils in the pathogenesis of idiopathic pulmonary fibrosis. Chest. 1997 Nov. 5; 112(5):1338-43. doi: 10.1378/chest.112.5.1338. PMID: 9367478.
38. Hoskin T S, Crowther J M, Cheung J, et al. Oxidative cross-linking of calprotectin occurs in vivo, altering its structure and susceptibility to proteolysis. Redox Biol. 2019; 24:101202. doi:10.1016/j.redox.2019.101202
39. Greten F R, Grivennikov S I. Inflammation and Cancer: Triggers, Mechanisms, and Consequences. Immunity. 2019 Jul. 16; 51(1):27-41. doi: 10.1016/j.immuni.2019.06.025. PMID: 31315034; PMCID: PMC6831096.

1: An immunoassay method for detecting an HNE-generated fragment of calprotectin, said method comprising contacting a human biofluid sample with a monoclonal antibody that specifically recognises and binds to an HNE-generated neo-epitope consisting of an N-terminus or C-terminus sequence of the HNE-generated fragment of calprotectin, and detecting binding between the monoclonal antibody and peptides in the sample.

2: The method as claimed in claim 1, wherein the detection is quantitative, and wherein the method further comprises determining the amount of binding between said monoclonal antibody and peptides in the sample.

3: The method as claimed in claim 2, wherein the method is an immunoassay method for detecting and/or monitoring the progress of and/or determining the status or severity of a disease in a patient, wherein the disease is a disease characterized by or exhibiting inflammation, the method comprising contacting a biofluid sample obtained from said patient with the monoclonal antibody, detecting and determining the amount of binding between the monoclonal antibody and peptides in the sample, and correlating said amount of binding with values associated with normal healthy subjects and/or with values associated with a known status or severity of the disease and/or with values obtained from said patient at a previous time point and/or with a predetermined cut-off value.

4: The method as claimed in claim 3, wherein the disease is an inflammatory driven disease.

5: The method as claimed in claim 4, wherein the disease is inflammatory bowel disease (IBD), rheumatoid arthritis, psoriasis, psoriasis arthritis, ankylosing spondylitis, osteoarthritis.

6: The method as claimed in claim 3, wherein the disease is chronic obstructive pulmonary disease (COPD) or idiopathic pulmonary fibrosis (IPF).

7: The method as claimed in claim 3, wherein the disease is a cancer.

8: The method as claimed in claim 7, wherein the disease is metastatic melanoma, small cell lung cancer (SCLC) or non-small cell lung cancer (NSCLC).

9: The method as claimed in claim 1, wherein the monoclonal antibody is a monoclonal antibody raised against a synthetic peptide comprising said N-terminus or C-terminus sequence.

10: The method as claimed in claim 1, wherein the monoclonal antibody specifically recognises and binds to an N-terminus or C-terminus sequence of a peptide selected from one of the following HNE-generated fragments of calprotectin:
(SEQ ID NO: 1)
KLGHPDTLNQGEFKELV,
(SEQ ID NO: 2)
RKDLQNFLKKENKNEKV,
(SEQ ID NO: 3)
RKDLQNFLKKENKNEKVI,
(SEQ ID NO: 4)
EHIMEDLDTNADKQL,
(SEQ ID NO: 5)
SHEKMHEGDEGPGHHHKPGLGEGTP,
(SEQ ID NO: 6)
YRDDLKKLLET,
and
(SEQ ID NO: 7)
WFKELDINTDGAV.

11: The method as claimed in claim 1, wherein the monoclonal antibody specifically recognises and binds to an N-terminus or C-terminus sequence of the peptide KLGHPDTLNQGEFKELV (SEQ ID NO: 1).

12: The method as claimed in claim 1, wherein the monoclonal antibody specifically recognises and binds to said N-terminus sequence of the HNE-generated fragment of calprotectin and does not specifically recognise or bind to an N-extended elongated version of said N-terminus amino acid sequence or an N-truncated shortened version of said N-terminus amino acid sequence.

13: The method as claimed in claim 1, wherein the biofluid sample is plasma or serum.

14: The method as claimed in claim 1, wherein the immunoassay is a competitive immunoassay.

15: The method as claimed in claim 1, wherein the immunoassay is an enzyme-linked immunosorbent assay (ELISA).

16: A monoclonal antibody that specifically recognises and binds to an HNE-generated neo-epitope consisting of an N-terminus or C-terminus sequence of an HNE-generated fragment of calprotectin.

17: The monoclonal antibody as claimed in claim 16, wherein the monoclonal antibody is a monoclonal antibody raised against a synthetic peptide comprising said N-terminus or C-terminus sequence.

18: The monoclonal antibody as claimed in claim 16, wherein the monoclonal antibody specifically recognises and binds to an N-terminus or C-terminus sequence of a peptide selected from one of the following HNE-generated fragments of calprotectin:
(SEQ ID NO: 1)
KLGHPDTLNQGEFKELV,
(SEQ ID NO: 2)
RKDLQNFLKKENKNEKV,
(SEQ ID NO: 3)
RKDLQNFLKKENKNEKVI,
(SEQ ID NO: 4)
EHIMEDLDTNADKQL,
(SEQ ID NO: 5)
SHEKMHEGDEGPGHHHKPGLGEGTP,
(SEQ ID NO: 6)
YRDDLKKLLET,
and
(SEQ ID NO: 7)
WFKELDINTDGAV.

19: The monoclonal antibody as claimed in claim 16, wherein the monoclonal antibody specifically recognises and binds to an N-terminus or C-terminus sequence of the peptide
(SEQ ID NO: 1)
KLGHPDTLNQGEFKELV.

20: The monoclonal antibody as claimed in claim 16, wherein the monoclonal antibody specifically recognises and binds to said N-terminus sequence of the HNE-generated fragment of calprotectin and does not specifically recognise or bind to an N-extended elongated version of said N-terminus amino acid sequence or an N-truncated shortened version of said N-terminus amino acid sequence.

21: An immunoassay kit comprising a monoclonal antibody as claimed in claim 16, and at least one of:
a streptavidin coated well plate,
a biotinylated peptide comprising said N-terminus or C-terminus sequence linked to biotin,
a secondary antibody for use in a sandwich immunoassay,
a calibrator peptide comprising said N-terminus or C-terminus sequence,
an antibody biotinylation
an antibody HRP labelling kit, or
an antibody radiolabelling kit.

22: The assay kit as claimed in claim 21, wherein the immunoassay kit comprises the monoclonal antibody, and one, two or all of:
a streptavidin coated well plate,
a biotinylated peptide comprising said N-terminus or C-terminus sequence linked to biotin, or
a calibrator peptide comprising said N-terminus or C-terminus sequence.
